(19) United States
(12) Patent Application Publication
(10) Pub. No.: US 2024/0401113 A1
(43) Pub. Date: Dec. 5, 2024

(54) AMPLIFICATION SYSTEMS FOR NANO-PLASMONIC MOLECULAR PROBES AND METHODS THEREOF

(71) Applicant: Duke University, Durham, NC (US)

(72) Inventors: Tuan Vo-Dinh, Durham, NC (US); Hsin-Neng Wang, Durham, NC (US)

(21) Appl. No.: 18/679,556

(22) Filed: May 31, 2024

Related U.S. Application Data
(60) Provisional application No. 63/470,234, filed on Jun. 1, 2023.

(51) Int. Cl. C12Q 1/6823; C12Q 1/6837

(52) U.S. Cl. CPC C12Q 1/6823; C12Q 1/6837; C12Q 2600/178

(57) ABSTRACT

To overcome the limitations of existing methods for detecting short nucleic acid molecules of low abundance such as miRNA, the present inventors provide a non-enzymatic signal amplification method based on inverse molecular sentinel (iMS) nanoprobes to improve detection sensitivity. The method is based on a cascade toehold-mediated DNA strand displacement reaction triggered by a “linear” DNA strand called “Recycling Trigger Probe” (RTP) strand. In the method, iMS-OFF nanoprobes are incubated with targets and RTP strands. After turning on the first nanoprobe, the target undergoes a recycling process triggered by the RTP strands. This process allows the target to turn on more iMS nanoprobes and provide an amplified SERS signal.

### CROSS-REFERENCE TO RELATED APPLICATIONS

This application claims priority to U.S. Provisional Patent Application No. 63/470,234 filed on Jun. 1, 2023, which is incorporated by reference herein in its entirety.

### SEQUENCE LISTING

The instant application contains a Sequence Listing which has been submitted electronically in XML file format and is hereby incorporated by reference in its entirety. Said XML copy, created on May 29, 2024, is named 400_51_UTIL_SL.xml and is 10,806 bytes in size.

### BACKGROUND

MicroRNAs (miRNAs), which are short, noncoding endogenous RNA molecules, have emerged as important diagnostic biomarkers and therapeutic targets for a variety of diseases. These small RNAs can bind to their mRNA targets in the untranslated regions (UTRs) and regulate gene expression at the post-transcriptional level. Over the past years, it has been demonstrated that miRNAs play an essential role in the regulation of various biological processes including oncogenesis. They can function both as tumor suppressors and as oncogenes. For example, miRNAs can transcriptionally suppress the expression of oncogenes and loss of miRNA expression, which results in oncogene activation. Recent studies have also shown that the expression profiles of miRNAs are dysregulated in many diseases, including cancer, infectious diseases, cardiovascular diseases, etc. In particular, miR-21, one of the most intensively studied miRNAs, has been described as an oncomiR, which is overexpressed in many cancers, including the aggressive triple-negative breast cancer, esophageal adenocarcinoma and colorectal cancer. Moreover, it has been shown that miR-21, together with other miRNAs, not only can be used as a signature to distinguish cancer and healthy samples but also can serve as predictors for overall and relapse-free survival. Therefore, miRNAs have been recognized as an important class of biomarkers for cancer early diagnosis, prognosis, and treatment. Rapid and accurate measurement of miRNA expression levels is critical for the evaluation of cancer risk, early detection, and the assessment of treatment efficacy. However, these small molecules have not been adopted into clinical practice because of the technical difficulties arising from the intrinsic characteristics of miRNAs, such as the short sequence lengths, low abundance, high sequence similarity, and a wide range of expression levels that can span over four orders of magnitude.

Advances in nucleic acid-based detection have provided important tools in molecular diagnostics because of their high specificity and sensitivity. The conventional methods for miRNA detection include northern blotting, quantitative real-time PCR (qRT-PCR), and microarrays. In the case of northern blotting, radiolabeling is involved, thus requiring sophisticated equipment from research labs and trained personnel to run the assays. Target amplification-based assays such as qRT-PCR and microarrays have costly reagents and instrumentation requirements, severely limiting their widespread adoption for diagnostics in clinical or at point-of-care (POC). In recent years, a variety of alternative miRNA detection strategies, including isothermal amplification, lateral flow assays, electrochemical-based systems, microfluidic chips, and nanotechnology sensing systems, have been developed. However, the sensitivity and specificity of these techniques remain challenging due to the aforementioned difficulties for the detection of miRNAs in biological samples.

Over the past years, surface-enhanced Raman scattering (SERS) has attracted increasing interest for use in molecular diagnostics owing to its ultra-high sensitivity and selectivity, providing superior diagnostic accuracy and multiplexed capability. SERS is a nanoscale optical phenomenon that arises mainly from the electromagnetic mechanism. The electromagnetic enhancement of SERS occurs when a light beam irradiates a metallic nanostructure. This process induces the collective oscillation of the free conduction electrons, called “surface plasmon”, resulting in a strong localized electromagnetic field around the nanostructures. In recent years, advances in nanotechnology and nanofabrication have led to the development of various plasmonic-active nanostructures and nanoparticles which exhibit enormous Raman signal enhancement on the order 10⁶-10⁷. The enhancement can even be up to 10¹⁵ through the so-called plasmonic coupling effect between two or more closely separated nanoparticles. For this reason, SERS has been long recognized as a powerful tool in the development of plasmonic-active biosensing platforms for a wide variety of applications, including biomedical diagnostics. Particularly, SERS-active nanoparticles have allowed for the achievement of sensitive and specific detection of nucleic acid biomarkers by utilizing the molecularly specific fingerprints provided by SERS. It also offers excellent multiplex capabilities, allowing for simultaneous detection of multiple targets due to the narrow spectral bandwidths of Raman peaks.

In recent years, various signal amplification strategies have been developed for miRNA detection instead of using target amplification schemes like PCR. To achieve high sensitivity, many of the detection methods employ enzyme-assisted amplification strategies allowing to recycle and reuse the target. Generally, in these methods, the enzyme, such as Exonuclease III (Exo III), duplex-specific nuclease (DSN), or DNase I, is used to cleave or degrade only the probe strand when the probe hybridizes to its target. The target is then released to trigger a series of cascaded enzymatic reactions for signal amplification. While these enzymatic strategies can effectively improve the detection sensitivity, there are still many limitations when using enzymes as their activity is dependent on various reaction conditions (i.e., reaction temperature, buffer solution, reaction time). These limitations can lead to false positive results.

Accordingly, there remains an unmet need for signal amplification strategies that are sensitive enough to detect short nucleic acid molecules of low abundance such as miRNA.

### SUMMARY

In a first aspect of the invention, a plasmonic-active nanoprobe system for detecting a target nucleic acid is provided. The system comprising: a) at least one plasmonic-active nanoparticle; b) a nucleic acid probe comprising a sequence that forms a stem loop, a first end attached to the at least one plasmonic-active nanoparticle and a second end labeled with an optical reporter; c) a recycling trigger nucleic acid probe (RTP); and d) a placeholder nucleic acid strand comprising: i) at one end a “toehold-1” region comprising complementarity to a sequence in a target nucleic acid, ii) at the other end a “toehold-2” region comprising complementarity to a sequence in the RTP, and iii) a third region between the “toehold-1” and toehold-2 regions complementary to the sequence in the nucleic acid probe that forms a stem-loop or a portion thereof. When the placeholder nucleic acid strand is hybridized with the nucleic acid probe, the optical reporter is at a greatest distance from the plasmonic-active nanoparticle and the nucleic acid probe is turned OFF. In the presence of the target nucleic acid, the placeholder nucleic acid strand binds to the sequence in the target nucleic acid, thereby forming a target/placeholder duplex and releasing the nucleic acid probe. The stem loop of the nucleic acid probe closes, thereby inducing an optical signal from the optical reporter and the nucleic acid probe is turned ON. The RTP strand binds to the toehold-2 domain of the target/placeholder duplex, thereby forming a placeholder/RTP duplex and releasing the target nucleic acid from the target/placeholder duplex for recycling.

In a second aspect of the invention, a method for detecting a target nucleic acid is provided. The method comprising first contacting a sample with a plasmonic-active nanoprobe system, the system comprising: a) at least one plasmonic-active nanoparticle, b) a nucleic acid probe comprising a sequence that forms a stem loop, a first end attached to the at least one plasmonic-active nanoparticle, and a second end labeled with an optical reporter, c) a recycling trigger nucleic acid probe (RTP), and d) a placeholder nucleic acid strand comprising: i) at one end a “toehold-1” region comprising complementarity to a sequence in a target nucleic acid, ii) at the other end a “toehold-2” region comprising complementarity to a sequence in the RTP, and iii) a third region between the “toehold-1” and toehold-2 regions complementary to the sequence in the nucleic acid probe that forms a stem-loop or a portion thereof. When the placeholder nucleic acid strand is hybridized with the nucleic acid probe, the optical reporter is at a greatest distance from the plasmonic-active nanoparticle and the nucleic acid probe is turned OFF. The method comprises detecting an optical signal from the optical reporter in the presence of the target nucleic acid. Specifically, in the presence of the target nucleic acid, the placeholder nucleic acid strand binds to the sequence in the target nucleic acid, thereby forming a target/placeholder duplex and releasing the nucleic acid probe. The stem loop of the nucleic acid probe closes, thereby inducing an optical signal from the optical reporter and the nucleic acid probe is turned ON. The RTP strand binds to the toehold-2 domain of the target/placeholder duplex, thereby forming a placeholder/RTP duplex and releasing the target nucleic acid from the target/placeholder duplex for recycling.

In a third aspect of the invention, a plasmonic-active nanoprobe system for detecting a target nucleic acid is provided. The system comprising: a) at least one plasmonic-active nanoparticle; b) a nucleic acid probe comprising a sequence that forms a stem loop, a first end attached to the at least one plasmonic-active nanoparticle, and a second end labeled with an optical reporter; c) a recycling trigger nucleic acid probe (RTP); d) a first placeholder nucleic acid strand comprising: i) at one end, a first region and a toehold-1 region, each of which is complementary to a sequence in a target nucleic acid, ii) an internal toehold-2 region complementary to a sequence in the RTP, and iii) a fourth region at the opposite end to the first region, complementary to the sequence in the nucleic acid probe that forms a stem-loop or a portion thereof; and e) a second placeholder nucleic acid strand having a region complementary to the toehold-2 region of the first placeholder and a region complementary to the first region of the first placeholder and excluding complementarity to the toehold-1 region. When the first placeholder nucleic acid strand is hybridized with the nucleic acid probe and the second placeholder nucleic acid strand is hybridized with the first placeholder nucleic acid strand, the nucleic acid probe is turned OFF. In the presence of the target nucleic acid, the target binds to the toehold-1 region and thereby replaces the second placeholder nucleic acid strand. The internal toehold-2 region of the first placeholder becomes single-stranded and the RTP strand binds to the toehold-2 region thereby releasing the first placeholder nucleic acid strand from the nucleic acid probe. The stem loop of the nucleic acid probe closes, thereby inducing an optical signal from the optical reporter, turning the nucleic acid probe ON, and releasing the target strand for recycling.

In a fourth aspect of the invention, a method for the detection of a target nucleic acid in a sample is provided. The method comprising contacting a sample with a plasmonic-active nanoprobe system, the system comprising: a) at least one plasmonic-active nanoparticle; b) a nucleic acid probe comprising a sequence that forms a stem loop, a first end attached to the at least one plasmonic-active nanoparticle, and a second end labeled with an optical reporter; c) a recycling trigger nucleic acid probe (RTP); d) a first placeholder nucleic acid strand comprising: i) at one end, a first region and a toehold-1 region, each of which are complementary to a sequence in a target nucleic acid, ii) an internal toehold-2 region complementary to a sequence in the RTP, and iii) a fourth region at the opposite end to the first region, complementary to the sequence in the nucleic acid probe that forms a stem-loop or a portion thereof, and e) a second placeholder nucleic acid strand having a region complementary to the toehold-2 region of the first placeholder and a region complementary to the first region of the first placeholder and excluding complementarity to the toehold-1 region. When the first placeholder nucleic acid strand is hybridized with the nucleic acid probe and the second placeholder nucleic acid strand is hybridized with the first placeholder nucleic acid strand, the nucleic acid probe is turned OFF. The method comprises detecting an optical signal from the optical reporter in the presence of the target nucleic acid. Specifically, in the presence of the target nucleic acid, the target binds to the toehold-1 region and thereby replaces the second placeholder nucleic acid strand. The internal toehold-2 region of the first placeholder becomes single-stranded and the RTP strand binds to the toehold-2 region thereby releasing the first placeholder nucleic acid strand from the nucleic acid probe. The stem loop of the nucleic acid probe closes, thereby inducing an optical signal from the optical reporter turning the nucleic acid probe ON and releasing the target strand for recycling.

In a fifth aspect of the invention, a plasmonic-active nanoprobe system for detecting a target protein is provided. The system, comprising: a) at least one plasmonic-active nanoparticle; b) a nucleic acid probe comprising a sequence that forms a stem loop, a first end attached to the at least one plasmonic-active nanoparticle, and a second end labeled with an optical reporter; c) a recycling trigger nucleic acid probe (RTP); and d) an aptamer placeholder nucleic acid strand comprising: i) at one end a first region comprising a “toehold-1” portion and a portion having a secondary structure that can bind to a target protein, ii) at the other end a “toehold-2” region comprising complementarity to a sequence in the RTP, and iii) a third region between the first and toehold-2 regions complementary to the sequence in the nucleic acid probe that forms a stem-loop or a portion thereof. When the aptamer placeholder strand is hybridized with the nucleic acid probe, the optical reporter is at a greatest distance from the plasmonic-active nanoparticle and the nucleic acid probe is turned OFF. In the presence of the target protein, the aptamer placeholder strand binds to the target protein, thereby forming a protein/aptamer complex and releasing the nucleic acid probe. The stem loop of the nucleic acid probe closes, thereby inducing an optical signal from the optical reporter and the nucleic acid probe is turned ON. The RTP strand binds to the toehold-2 domain of the protein/aptamer complex, thereby forming an aptamer placeholder/RTP duplex and releasing the target protein from the protein/aptamer complex for recycling.

In a sixth aspect of the invention, a method for detection of a target protein in a sample is provided. The method comprising: contacting a sample with a plasmonic-active nanoprobe system, the system comprising: i) at least one plasmonic-active nanoparticle, ii) a nucleic acid probe comprising a sequence that forms a stem loop, a first end attached to the at least one plasmonic-active nanoparticle, and a second end labeled with an optical reporter, iii) a recycling trigger nucleic acid probe (RTP), and iv) an aptamer placeholder nucleic acid strand. The aptamer nucleic acid strand comprises at one end a first region comprising a “toehold-1” portion and a portion having a secondary structure that can bind to a target protein, at the other end a “toehold-2” region comprising complementarity to a sequence in the RTP, and a third region between the first and toehold-2 regions complementary to the sequence in the nucleic acid probe that forms a stem-loop or a portion thereof. When the aptamer placeholder strand is hybridized with the nucleic acid probe, the optical reporter is at a greatest distance from the plasmonic-active nanoparticle and the nucleic acid probe is turned OFF. The method comprises detecting an optical signal from the optical reporter in the presence of the target protein, wherein, in the presence of the target protein, the aptamer placeholder strand binds to the target protein, thereby forming a protein/aptamer complex and releasing the nucleic acid probe. The stem loop of the nucleic acid probe closes, thereby inducing an optical signal from the optical reporter and the nucleic acid probe is turned ON. The RTP strand binds to the toehold-2 domain of the protein/aptamer complex, thereby forming an aptamer placeholder/RTP duplex and releasing the target protein from the protein/aptamer complex for recycling.

In a seventh aspect of the invention, a kit is provided for detecting a target nucleic acid, the kit comprising a plasmonic-active nanoprobe system. The plasmonic-active nanoprobe system comprises a) at least one plasmonic-active nanoparticle; b) a nucleic acid probe comprising a sequence that forms a stem loop, a first end attached to the at least one plasmonic-active nanoparticle, and a second end labeled with an optical reporter; c) a recycling trigger nucleic acid probe (RTP); d) a placeholder nucleic acid strand comprising: i) at one end a toehold-1 region comprising complementarity to a sequence in a target nucleic acid, ii) at the other end a “toehold-2” region comprising complementarity to a sequence in the RTP, and iii) a third region between the toehold-1 and toehold-2 regions complementary to the sequence in the nucleic acid probe that forms a stem-loop or a portion thereof; and e) directions for contacting a sample with the plasmonic-active nanoprobe system and detecting an optical signal from the optical reporter in the presence of the target nucleic acid.

In an eighth aspect of the invention, a kit is provided for detecting a target nucleic acid, the kit comprising a plasmonic-active nanoprobe system. The plasmonic-active nanoprobe system comprises: a) at least one plasmonic-active nanoparticle; b) a nucleic acid probe comprising a sequence that forms a stem loop, a first end attached to the at least one plasmonic-active nanoparticle, and a second end labeled with an optical reporter; c) a recycling trigger nucleic acid probe (RTP); d) a first placeholder nucleic acid strand comprising: i) at one end, a first region and a toehold-1 region, each of which are complementary to a sequence in a target nucleic acid, ii) an internal toehold-2 region complementary to a sequence in the RTP, and iii) a fourth region at the opposite end to the first region, complementary to the sequence in the nucleic acid probe that forms a stem-loop or a portion thereof; e) a second placeholder nucleic acid strand having a region complementary to the toehold-2 region of the first placeholder and a region complementary to the first region of the first placeholder and excluding complementarity to the toehold-1 region; and f) directions for contacting a sample with the plasmonic-active nanoprobe system and detecting an optical signal from the optical reporter in the presence of the target nucleic acid.

In a ninth aspect of the invention, a kit is provided for detecting a protein target. The kit comprising a plasmonic-active nanoprobe system, comprising: a) at least one plasmonic-active nanoparticle; b) a nucleic acid probe comprising a sequence that forms a stem loop, a first end attached to the at least one plasmonic-active nanoparticle, and a second end labeled with an optical reporter; c) a recycling trigger nucleic acid probe (RTP); d) an aptamer placeholder nucleic acid strand comprising: i) at one end a first region comprising a “toehold-1” portion and a portion having a secondary structure that can bind to a target protein, ii) at the other end a “toehold-2” region comprising complementarity to a sequence in the RTP, and iii) a third region between the first and toehold-2 regions complementary to the sequence in the nucleic acid probe that forms a stem-loop or a portion thereof; and e) directions for contacting a sample with the plasmonic-active nanoprobe system and detecting an optical signal from the optical reporter in the presence of the target protein.

### BRIEF DESCRIPTION OF THE DRAWINGS

The accompanying Figures and Examples are provided by way of illustration and not by way of limitation. The foregoing aspects and other features of the disclosure are explained in the following description, taken in connection with the accompanying example figures (also “FIG.”) relating to one or more embodiments.

FIG. 1 is a schematic diagram showing the OFF/ON mechanism of an iMS nanoprobe comprising a plasmonics-active nanostar, a Raman-labeled stem-loop DNA probe, and a placeholder strand.

FIG. 2A is a schematic diagram showing the detection of miRNA targets using the Cascade Amplification by Recycling Trigger Probe (CARTP) strategy for iMS SERS signal amplification when the iMS are in a solution.

FIG. 2B is a schematic diagram showing the detection of miRNA targets using the CARTP strategy on a SERS substrate or chip for iMS SERS signal amplification when the iMS are bound to a chip substrate.

FIG. 3A is a schematic diagram depicting the amplified iMS detection strategy for nucleic acid targets.

FIG. 3B is a schematic diagram depicting the amplified iMS detection strategy for protein targets.

FIG. 4 is a schematic diagram showing the cascade iMS amplification scheme based on the CARTP method.

FIG. 5 is a schematic diagram illustrating the preparation process for the synthesis of the iMS nanoprobes.

FIG. 6A shows the sequence structures of probe/placeholder duplex showing the sequences of toehold-1.

FIG. 6B shows the sequence structures of placeholder/target duplex showing the sequences of toehold-2.

FIG. 7A is a graph showing representative SERS spectra of the iMS-nanoprobes with Placeholder-1 in the presence of 100 nM RTP strands. The nanoprobes were incubated with 1 nM miR-21 targets (Blue spectrum: Target (+)) or without targets (Orange spectrum: Blank) for 24 hours at room temperature. The arrow indicates the 557-cm⁻¹ peak of the Raman label (Cy5) conjugated on the DNA probes. The spectra were taken using 4.8-mW laser power, 10-second exposure time and 5 accumulations.

FIG. 7B is a graph showing the blank-subtracted SERS signal corresponding to FIG. 7A.

FIG. 8A is a graph showing a blank-subtracted SERS peak-height intensity at 557 cm⁻¹ of the miR-21 iMS nanoprobes with Placeholder-1 in the presence (denoted as RTP (+)) or absence (denoted as RTP (−)) of 100 nM RTP strands. The SERS signal was measured after 3- or 24-hour incubation at room temperature with 1 nM targets (denoted as Target (+)). After incubation, 100 μL of the samples were transferred to a glass vial for SERS measurements. The spectra were taken using 4.8-mW laser power, 10-second exposure time and 5 accumulations.

FIG. 8B is a graph showing a blank-subtracted SERS peak-height intensity at 557 cm⁻¹ of the miR-21 iMS nanoprobes with Placeholder-2 in the presence (denoted as RTP (+)) or absence (denoted as RTP (−)) of 100 nM RTP strands. The SERS signal was measured after 3- or 24-hour incubation at room temperature with 1 nM targets (denoted as Target (+)). After incubation, 100 μL of the samples were transferred to a glass vial for SERS measurements. The spectra were taken using 4.8-mW laser power, 10-second exposure time and 5 accumulations.

FIG. 8C is a graph showing a blank-subtracted SERS peak-height intensity at 557 cm⁻¹ of the miR-21 iMS nanoprobes with Placeholder-3 in the presence (denoted as RTP (+)) or absence (denoted as RTP (−)) of 100 nM RTP strands. The SERS signal was measured after 3- or 24-hour incubation at room temperature with 1 nM targets (denoted as Target (+)). After incubation, 100 μL of the samples were transferred to a glass vial for SERS measurements. The spectra were taken using 4.8-mW laser power, 10-second exposure time and 5 accumulations.

FIG. 9A is a graph showing a blank-subtracted SERS peak-height intensity at 557 cm⁻¹ of the miR-21 iMS nanoprobes with Placeholder-1 incubated with 0.05, 0.1, 1 and 10 μM synthetic targets for 24 hours at room temperature in the presence of 100 nM RTP strands for SERS signal amplification. After incubation, 5 μL of the samples were transferred to a glass capillary tube for SERS measurements. The spectra were taken using 7.3-mW laser power, 10-second exposure time and 3 accumulations.

FIG. 9B is a graph showing a blank-subtracted SERS peak-height intensity at 557 cm⁻¹ of the miR-21 iMS nanoprobes with Placeholder-2 incubated with 0.05, 0.1, 1 and 10 μM synthetic targets for 24 hours at room temperature in the presence of 100 nM RTP strands for SERS signal amplification. After incubation, 5 μL of the samples were transferred to a glass capillary tube for SERS measurements. The spectra were taken using 7.3-mW laser power, 10-second exposure time and 3 accumulations.

FIG. 10 is a graph showing a normalized standard curve for the evaluation of the miR-21 detection sensitivity using the iMS nanoprobes with the CARTP strategy. The iMS nanoprobes with Placeholder-1 were incubated with 0 (blank), 25, 50, 100 and 200 fM synthetic targets for 24 hours at room temperature in the presence of 100 nM RTP strands. After incubation, 5 μL of the samples were transferred to a glass capillary tube for SERS measurements. The spectra were taken using 7.3-mW laser power, 10-second exposure time and 3 accumulations. The SERS intensities were then blank subtracted with the average blank signal and normalized to the highest signal from the measurements.

FIG. 11 is a schematic diagram of an inverse molecular sentinel nanosensor.

FIG. 12 is a schematic diagram showing RTP-mediated target recycling and amplification where the RTP strand includes a stem-loop (hairpin) to prevent non-specific interaction between the RTP strand and the probe/placeholder duplex.

FIG. 13 is a schematic diagram showing RTP-mediated target recycling and amplification where the placeholder is designed as a hairpin strand with the stem duplex a1+a2/a1′+a2′ to prevent the non-specific interaction with the RTP strand.

FIG. 14A is a schematic diagram showing a potential issue with RTP-mediated target recycling and amplification when using a linear RTP strand.

FIG. 14B is a schematic diagram showing a toehold-locking strategy to prevent the released target from interacting with the placeholder/RTP duplex as illustrated in FIG. 14A by using a “Locker” strand.

FIG. 15 is a schematic diagram showing RTP-mediated target recycling and amplification using a linear RTP strand and two placeholders.

FIG. 16A is a schematic diagram of an iMS amplification system involving chain reaction probes (CRP) to amplify the SERS signal initially produced by the recognition of the target by the iMS probe showing how the Target encounters the iMS probe that has a placeholder and a trigger probe.

FIG. 16B is a schematic diagram of the iMS amplification system of FIG. 16A showing how the Target next hybridizes and removes the placeholder from the iMS probe.

FIG. 16C is a schematic diagram of the iMS amplification system of FIG. 16B showing how the Target iMS probe forms a stem loop, brings the Raman label close to the plasmonic surface (SERS on) and releases the trigger probe. This is the Initial SERS signal from iMS.

FIG. 16D is a schematic diagram of the iMS amplification system of FIG. 16C showing how the Chain Reaction Probe (CRP) 1 has a placeholder and a trigger probe.

FIG. 16E is a schematic diagram of the iMS amplification system of FIG. 16D showing how the Trigger probe (released from the iMS) hybridizes and removes the placeholder from CRP1.

FIG. 16F is a schematic diagram of the iMS amplification system of FIG. 16E showing how the CRP1 forms a stem loop, brings the Raman label close to the plasmonic surface (SERS+) and releases its trigger probe. This is the 1^{st} triggered SERS signal from CPR1.

FIG. 16G is a schematic diagram of the iMS amplification system of FIG. 16F showing how the released trigger probe from CRP1 encounters a CRP2 and hybridizes with the placeholder of CRP2.

FIG. 16H is a schematic diagram of the iMS amplification system of FIG. 16G showing how the CRP2 forms a stem loop, brings the Raman label close to the plasmonic surface (SERS++) and releases the trigger probe. This is the 2nd triggered SERS signal from CPR2 and the same chain reaction will continue with subsequent triggering of the SERS signal. The illustrated chain reaction will continue with CRP3, CPRn, etc.

FIG. 17 is a graph showing a blank-subtracted SERS peak-height intensity at 557 cm⁻¹ of the miR-142 iMS nanoprobes with the Placeholder in the presence (denoted as RTP (+)) or absence (denoted as RTP (−)) of 100 nM RTP strands. The SERS signal was measured after 1-hour incubation at 37° C. with 100 pM targets. After incubation, 100 μL of the samples were transferred to a glass vial for SERS measurements.

### DETAILED DESCRIPTION

To promote an understanding of the principles of the present disclosure, reference will now be made to preferred embodiments, and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or more than one (i.e., at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements.

As used herein, “and/or” refers to and encompasses any and all possible combinations of one or more of the associated listed items, as well as the lack of combinations when interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.” Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

As used herein, the term “microRNA” or “miRNA” is meant to include any nucleic acid having a sequence of 25 nucleotides or less. For example, the term “microRNA” can include a small noncoding RNA, an mRNA, or a DNA sequence, or combinations of any of these molecules.

As used herein, the phrases “placeholder nucleic acid strand”, “placeholder strand”, “placeholder”, and “placeholder DNA” are interchangeable.

As used herein, the terms “invader strand”, “fuel strand”, “Recycling Trigger Probe” (RTP) strand “, and “trigger probe” are interchangeable.

As used herein, the phrases “amplified iMS detection strategy”, “RTP-mediated target recycling and amplification”, and “Cascade Amplification by Recycling Trigger Probe (CARTP)” are interchangeable.

Recitation of ranges of values herein is merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered expressly stated in this disclosure. Unless otherwise defined, all technical terms used herein have the same meaning as commonly understood by one of ordinary skill in the art to which this disclosure belongs.

Described herein are methods and systems for detecting short nucleic acid molecules of low abundance, such as, but not limited to, miRNA. More specifically, the amplification systems described herein for detecting short nucleic acid sequences are based in part on a sensitive, specific, and multiplexed SERS-based detection scheme referred to as the “Inverse Molecular Sentinel” (iMS). The iMS sensing technique is a one-step homogeneous plasmonic nanobiosensor that uses a plasmonic-active nanoparticle as the sensing platform. In one embodiment, SERS-active silver-coated gold nanostars (AuNS@Ag) are used as the sensing platform. Gold nanostars (AuNS) have emerged as one of the best geometries for producing strong SERS in a non-aggregated state due to their multiple sharp branches, each with a strongly enhanced electromagnetic field localized at its tip. By coating them with silver (Ag), silver-coated gold nanostars (AuNS@Ag) have been demonstrated to offer over an order of magnitude of signal enhancement compared to uncoated AuNS. The iMS nanobiosensor described herein employs a non-enzymatic DNA strand-displacement process and the conformational change of stem-loop (hairpin) DNA probes for specific target identification and signal switch.

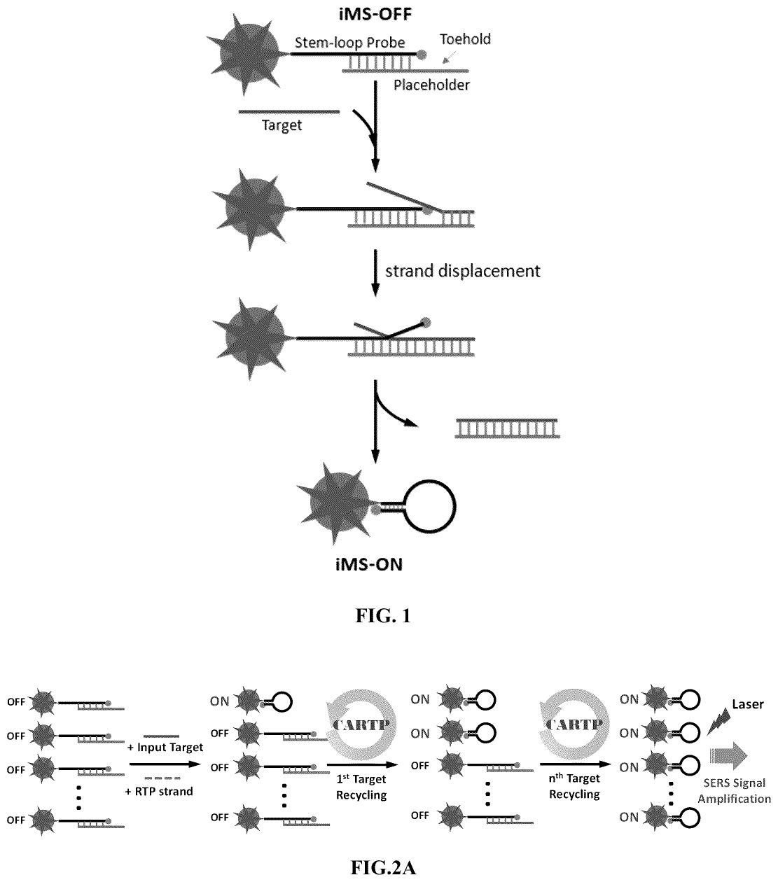

As shown in FIG. 1, an iMS-OFF nanoprobe comprises a plasmonics-active nanoparticle (e.g., a nanostar or a silver-coated gold nanostar (AuNS@Ag)), a labeled stem-loop DNA probe (e.g., Raman-labeled), and a placeholder strand. In this case, the stem-loop probe, having a Raman label at one end, is immobilized onto a nanostar via a metal-thiol bond. The probe is designed with a “stem-loop”, or “hairpin”, structure to produce a strong SERS signal when the loop is closed and brings the Raman label to the nanostar surface. The placeholder strand hybridizes to the stem-loop probe keeping the Raman dye away from the nanostar surface, thus turning the SERS signal “OFF” (iMS-OFF) as the SERS enhancement decreases exponentially with increasing separation between the label and the metallic surface. In the presence of a miRNA target (or another nucleic acid target), the target binds to the placeholder and displaces the stem-loop probe through a non-enzymatic toehold-mediated DNA strand displacement reaction. The displacement reaction is initiated by the binding of the target to the overhang region (called “toehold”) on the probe/placeholder duplex, followed by a branch migration process to displace the stem-loop probe from the placeholder. This process allows the placeholder to be released from the nanostar surface, leading to the formation of a “closed” stem-loop structure and switch the SERS signal “ON” by moving the Raman label onto the nanostar surface (IMS-ON).

In recent years, various signal amplification strategies have been developed for miRNA detection instead of using target amplification schemes like PCR. To achieve high sensitivity, many of the detection methods employ enzyme-assisted amplification strategies allowing to recycle and reuse the target. Generally, in these methods, the enzyme, such as Exonuclease III (Exo III), duplex-specific nuclease (DSN), or DNase I, is used to cleave or degrade only the probe strand when the probe hybridizes to its target. The target is then released to trigger a series of cascaded enzymatic reactions for signal amplification. While these enzymatic strategies can effectively improve the detection sensitivity, there are still many limitations when using enzymes as their activity is dependent on various reaction conditions (i.e., reaction temperature, buffer solution, reaction time). These limitations can lead to false positive results.

In addition, several alternative miRNA sensing strategies have been developed to facilitate the translation of miRNA biomarkers from basic research to clinical application. For example, it has been previously reported that when combining SERS and DNA strand displacement amplification, miRNA detection with LODs ranging from sub-fM to sub-pM can be achieved in 30 min to several hours. These SERS signal amplification assays provide wide dynamic ranges spanning five to eight orders of magnitude. However, many of these previously published methods require a sophisticated design of hairpin-structured strands to trigger the amplification process. Also, some of these methods require multiple washing/rinsing steps or magnetic separation. In other published approaches, anti-DNA/RNA antibodies have been used for the detection of miRNAs using other sensing methods, including Reflective Phantom Interface (RPI) technology, surface plasmon resonance imaging (SPRi) and enzyme-based amperometric sensing. These antibody-based assays generally provide LODs at sub-pM levels with dynamic ranges spanning three to four orders of magnitude. However, like enzymes, antibodies are more expensive and susceptible to various reaction and storage conditions compared to DNA. Moreover, these substrate-based assays require either rinsing steps or fabrication of different sensing spots to detect multiple targets.

Thus, there remains a need for simple but highly sensitive non-enzymatic (enzyme-free) signal amplification strategies for miRNA detection. One non-enzymatic signal amplification strategy is the toehold-mediated strand displacement (TMSD) amplification that utilizes the target strand as the trigger to initiate the amplification reaction. In this strategy, the target first hybridizes to a probe strand to create an overhang as the toehold. A third strand, commonly referred to as the “invader” or “fuel” strand, then binds to the probe at the toehold to initiate the strand displacement reaction, leading to the release of the target that can be reused for the next amplification cycle. While progress has been made in combining SERS and TMSD for miRNA detection, the TMSD-based detection strategies have unresolved challenges. One of the main issues is the non-specific interaction between the probe and the invader in the absence of a target, which increases the background signal, thus reducing the detection sensitivity. To overcome this issue, DNA hairpins are usually used as the invader to prevent non-specific interaction with the probe by hiding the complementary sequences inside the hairpin structure. However, it requires a sophisticated design of the hairpin invader to ensure the hairpin structure is sufficiently stable to prevent the non-specific interaction with the probe but not the strand migration of the target.

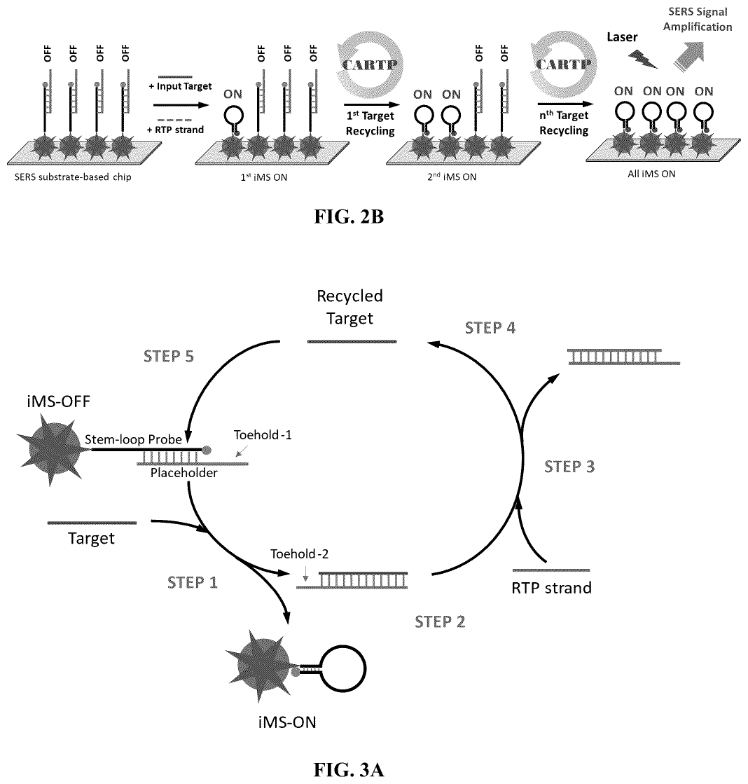

To overcome the limitations of existing methods for detecting short nucleic acid molecules of low abundance such as miRNA, the present inventors provide a simplified non-enzymatic iMS signal amplification strategy referred to as “Cascade Amplification by Recycling Trigger Probe” (CARTP), to improve the iMS detection sensitivity. This strategy is based on the cascade toehold-mediated DNA strand displacement reaction triggered by a “linear” DNA strand called “Recycling Trigger Probe” (RTP) strand. FIG. 2 schematically shows the detection of miRNA targets using the CARTP strategy for iMS SERS signal amplification. The CARTP strategy can be used when the iMS are in solution (FIG. 2A) or bound to a substrate-based or chip-based platform (FIG. 2B). In this strategy, the iMS-OFF nanoprobes are incubated with input targets and RTP strands. After turning on the first nanoprobe, the input target undergoes a recycling process triggered by the RTP strands. This process allows the target to turn on more iMS nanoprobes and provide an amplified SERS signal. The synthesis of SERS-active iMS nanoprobes and testing of the Cascade Amplification by Recycling Trigger Probe (CARTP) method provided herein is described in Example 1 and FIGS. 2-10.

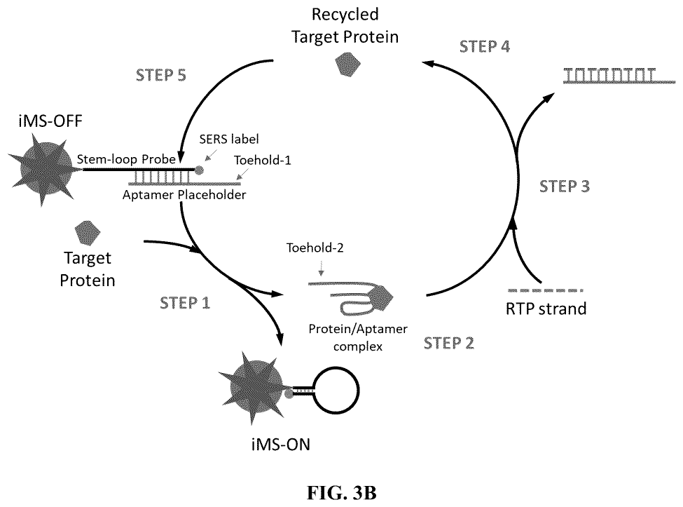

FIG. 3A depicts the amplified iMS detection strategy. In the presence of targets, the miRNA target binds to the toehold-1 to initiate the first strand displacement reaction and turn the SERS signal “ON” (STEP 1) by releasing the target/placeholder duplex (STEP 2). The released target/placeholder duplex then serves as a substrate for the RTP strand. The “linear” RTP strand can bind to the single-stranded overhang (toehold-2) of the target/placeholder duplex to trigger the second strand displacement reaction (STEP 3) allowing the target to be released from the target/placeholder duplex (STEP 4). In this way, the released target is recycled and reused to trigger the cascade DNA strand displacement reaction (STEP 5).

This CARTP method can also be applied for the detection of target protein using an aptamer as the placeholder. FIG. 3B depicts the amplified iMS detection strategy for target protein. In the presence of targets, the target protein binds to the toehold-1 and a portion of the aptamer to turn the SERS signal “ON” (STEP 1) by releasing the protein/aptamer complex (STEP 2). The released protein/aptamer then serves as a substrate for the RTP strand. The RTP strand can bind to the single-stranded overhang (toehold-2) of the protein/aptamer complex to trigger release of the target protein from the protein/aptamer complex (STEP 4). In this way, the released target protein is recycled and reused to trigger the cascade strand displacement reaction (STEP 5).

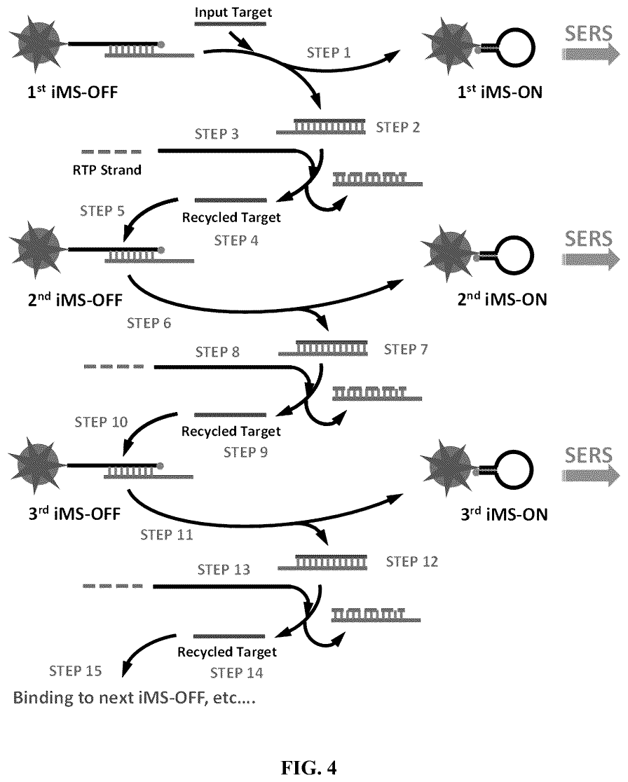

FIG. 4 shows the cascade amplification scheme based on the CARTP method after 3 cycles. After turning ON the 1st iMS-OFF nanoprobe, the input target is recycled at the end of each cycle (STEPS 4, 9 and 14) and subsequently turns ON more iMS-OFF nanoprobes (e.g., 2^{nd} iMS-OFF, 3rd iMS-OFF, and so on). Example 1, herein below, describes the detection of synthetic miR-21 targets with a significantly improved sensitivity, having a limit of detection (LOD) of 45 fM, which is 100-fold more sensitive than the non-amplified iMS assay reported previously.

In contrast to previously published methods, the amplified iMS detection strategy described herein is a homogeneous bioassay, which does not require PCR, target labeling, or any subsequent washing steps. The method provided herein can be used for iMS in solution or bound on a substrate for various applications. The multiplexed capability of the iMS sensing based on SERS also offers significant advantages over other optical methods, such as fluorescence and chemiluminescence. Multiplexed detection can be easily achieved in a one-pot format as multiple targets can be detected in a single solution or in the same spot on a SERS substrate by using different Raman labels. In addition, the method provided herein can be performed using a small sample volume (a few μL) in a capillary tube that could be advantageous for clinical applications when limited quantities of samples can be collected. In the amplified iMS detection strategy described herein, the nucleic acid target can include a microRNA, a small noncoding RNA, an mRNA, or a DNA sequence.

A plasmonic-active nanoprobe system for detecting a target nucleic acid is provided. The system comprising: a) at least one plasmonic-active nanoparticle; b) a nucleic acid probe comprising a sequence that forms a stem loop, c) a first end attached to the at least one plasmonic-active nanoparticle and a second end labeled with an optical reporter; d) a recycling trigger nucleic acid probe (RTP); and e) a placeholder nucleic acid strand comprising: i) at one end a toehold-1 region comprising complementarity to a sequence in a target nucleic acid, ii) at the other end a “toehold-2” region comprising complementarity to a sequence in the RTP, and iii) a third region between the toehold-1 and toehold-2 regions complementary to the sequence in the nucleic acid probe that forms a stem-loop or a portion thereof. When the placeholder nucleic acid strand is hybridized with the nucleic acid probe, the optical reporter is at a greatest distance from the plasmonic-active nanoparticle and the nucleic acid probe is turned OFF. In the presence of the target nucleic acid, the placeholder nucleic acid strand binds to the sequence in the target nucleic acid, thereby forming a target/placeholder duplex and releasing the nucleic acid probe. The stem loop of the nucleic acid probe closes, thereby inducing an optical signal from the optical reporter and the nucleic acid probe is turned ON. The RTP strand binds to the toehold-2 domain of the target/placeholder duplex, thereby forming a placeholder/RTP duplex and releasing the target nucleic acid from the target/placeholder duplex for recycling.

A method for detecting a target nucleic acid is provided. The method comprising first contacting a sample with a plasmonic-active nanoprobe system, the system comprising: a) at least one plasmonic-active nanoparticle, b) a nucleic acid probe comprising a sequence that forms a stem loop, a first end attached to the at least one plasmonic-active nanoparticle, and a second end labeled with an optical reporter, c) a recycling trigger nucleic acid probe (RTP), and d) a placeholder nucleic acid strand comprising: i) at one end a toehold-1 region comprising complementarity to a sequence in a target nucleic acid, ii) at the other end a “toehold-2” region comprising complementarity to a sequence in the RTP, and iii) a third region between the toehold-1 and toehold-2 regions complementary to the sequence in the nucleic acid probe that forms a stem-loop or a portion thereof. When the placeholder nucleic acid strand is hybridized with the nucleic acid probe, the optical reporter is at a greatest distance from the plasmonic-active nanoparticle and the nucleic acid probe is turned OFF. The method comprises detecting an optical signal from the optical reporter in the presence of the target nucleic acid. Specifically, in the presence of the target nucleic acid, the placeholder nucleic acid strand binds to the sequence in the target nucleic acid, thereby forming a target/placeholder duplex and releasing the nucleic acid probe. The stem loop of the nucleic acid probe closes, thereby inducing an optical signal from the optical reporter and the nucleic acid probe is turned ON. The RTP strand binds to the toehold-2 domain of the target/placeholder duplex, thereby forming a placeholder/RTP duplex and releasing the target nucleic acid from the target/placeholder duplex for recycling.

A plasmonic-active nanoprobe system for detecting a target protein is provided. The system, comprising: a) at least one plasmonic-active nanoparticle; b) a nucleic acid probe comprising a sequence that forms a stem loop, a first end attached to the at least one plasmonic-active nanoparticle, and a second end labeled with an optical reporter; c) a recycling trigger nucleic acid probe (RTP); and d) an aptamer placeholder nucleic acid strand comprising: i) at one end a first region comprising a “toehold-1” portion and a portion having a secondary structure that can bind to a target protein, ii) at the other end a “toehold-2” region comprising complementarity to a sequence in the RTP, and iii) a third region between the first and toehold-2 regions complementary to the sequence in the nucleic acid probe that forms a stem-loop or a portion thereof. When the aptamer placeholder strand is hybridized with the nucleic acid probe, the optical reporter is at a greatest distance from the plasmonic-active nanoparticle and the nucleic acid probe is turned OFF. In the presence of the target protein, the aptamer placeholder strand binds to the target protein, thereby forming a protein/aptamer complex and releasing the nucleic acid probe. The stem loop of the nucleic acid probe closes, thereby inducing an optical signal from the optical reporter and the nucleic acid probe is turned ON. The RTP strand binds to the toehold-2 domain of the protein/aptamer complex, thereby forming an aptamer placeholder/RTP duplex and releasing the target protein from the protein/aptamer complex for recycling.

A method for detection of a target protein in a sample is provided. The method comprising: contacting a sample with a plasmonic-active nanoprobe system, the system comprising: i) at least one plasmonic-active nanoparticle, ii) a nucleic acid probe comprising a sequence that forms a stem loop, a first end attached to the at least one plasmonic-active nanoparticle, and a second end labeled with an optical reporter, iii) a recycling trigger nucleic acid probe (RTP), and iv) an aptamer placeholder nucleic acid strand. The aptamer nucleic acid strand comprises at one end a first region comprising a “toehold-1” portion and a portion having a secondary structure that can bind to a target protein, at the other end a “toehold-2” region comprising complementarity to a sequence in the RTP, and a third region between the first and toehold-2 regions complementary to the sequence in the nucleic acid probe that forms a stem-loop or a portion thereof. When the aptamer placeholder strand is hybridized with the nucleic acid probe, the optical reporter is at a greatest distance from the plasmonic-active nanoparticle and the nucleic acid probe is turned OFF. The method comprises detecting an optical signal from the optical reporter in the presence of the target protein, wherein, in the presence of the target protein, the aptamer placeholder strand binds to the target protein, thereby forming a protein/aptamer complex and releasing the nucleic acid probe. The stem loop of the nucleic acid probe closes, thereby inducing an optical signal from the optical reporter and the nucleic acid probe is turned ON. The RTP strand binds to the toehold-2 domain of the protein/aptamer complex, thereby forming an aptamer placeholder/RTP duplex and releasing the target protein from the protein/aptamer complex for recycling.

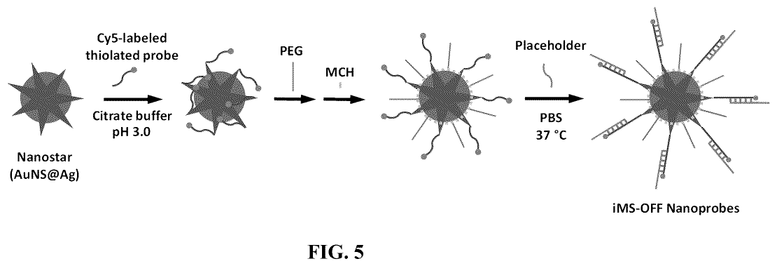

FIG. 5 shows a schematic diagram illustrating the preparation process for the synthesis of one embodiment of the iMS nanoprobes described herein. An experimental procedure for the preparation is detailed in Example 1. In one example, to prepare iMS-OFF nanoprobes, a solution of iMS DNA probe immobilized on silver-coated gold nanostars (iMS-AuNS@Ag) can be incubated with placeholder DNA for about 20 hours and the excess placeholder strands removed by washing. The iMS assay can be carried out by mixing the iMS—OFF-AuNS@Ag solution with miRNA targets of interest or a mixture of the miRNA targets and invaders. SERS measurements can be performed on the mixture using a Raman microscope equipped with a laser. The resulting SERS spectra can be background subtracted and smoothed. The number of labeled oligonucleotides immobilized on AuNS@Ag can be determined. The cascade iMS amplification is achieved through two toehold-mediated strand displacement reactions. To initiate the amplification process, the target strand binds to the toehold-1 domain on the probe/placeholder duplex for the first strand displacement reaction. To release the target, the RTP strand binds to the toehold-2 domain on the placeholder/target duplex for the second strand displacement reaction.

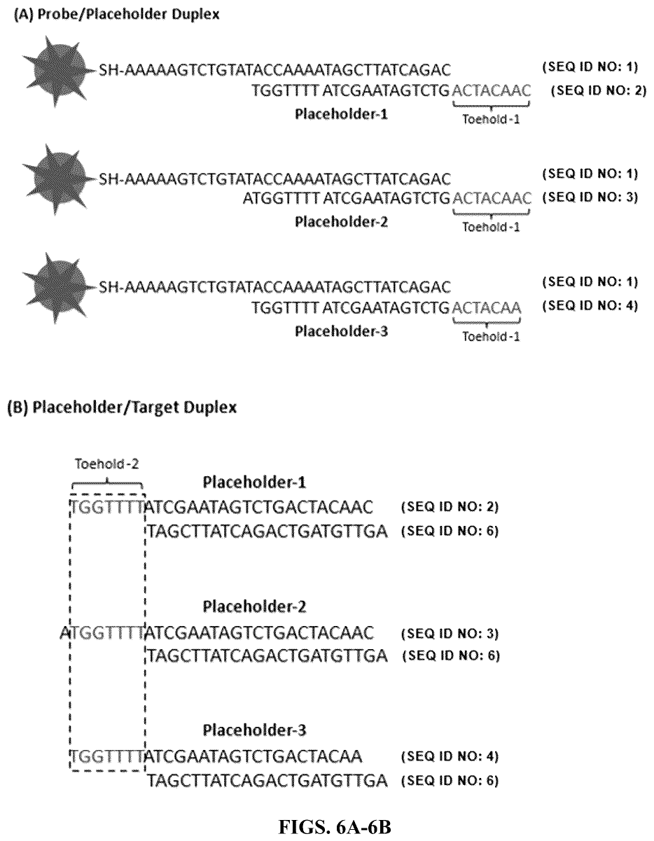

Tables 1 and 2 in Example 1 show exemplary sequences of the oligonucleotides that can be used in the methods provided herein for detecting two different nucleic acid targets, including thiolated Cy5-labeled stem-loop probes, placeholders, and RTP strands. In the study described in Example 1, three placeholder sequences are tested for the signal amplification strategy. FIG. 6A shows the sequence structures of probe/placeholder duplex showing the sequences of toehold-1. FIG. 6B shows the sequence structures of placeholder/target duplex showing the sequences of toehold-2. In this example, the stem-loop probe has two guanine (C) bases in the internal spacer instead of two adenine (A) bases as previously published. The Placeholder-2 has one additional base at the 3′ end to increase the stability of the probe/placeholder duplex.

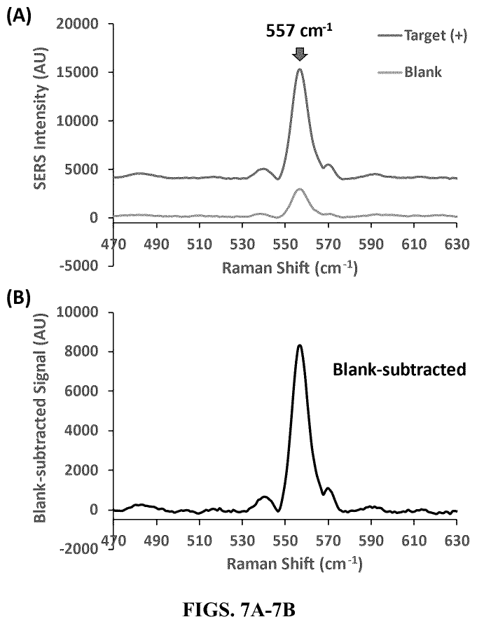

In the amplification strategy, the iMS-OFF nanoprobes can be incubated with targets. FIG. 7 presents an example of a representative SERS spectrum within the spectral region of the major peak of the iMS nanoprobes with Placeholder-1 in the presence of the RTP strand shown in Table 1. In FIG. 7, the iMS-OFF nanoprobes incubated with targets are denoted as Target (+) and the iMS-OFF nanoprobes incubated without targets are denoted as RTP (−). The arrow indicates the main Cy5 Raman peak at 557 cm⁻¹ for the peak-height intensity analysis in this study. As shown in FIG. 7A, the SERS intensity at 557 cm⁻¹ is significantly increased after incubation with miR-21 targets, indicating that a “closed” stem-loop probe structure was formed and the SERS signal was turned on upon the binding of the targets. Such a response can be seen in FIG. 7B, which shows the increased SERS intensity after blank subtraction.

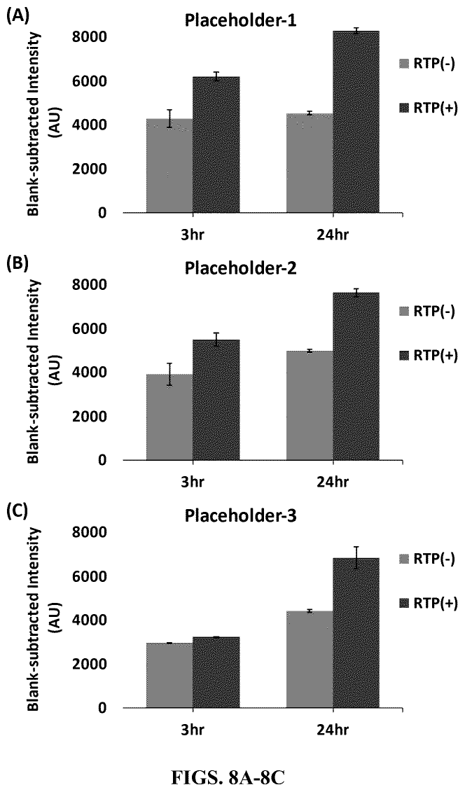

FIG. 8 shows that the blank-subtracted SERS peak-height intensities at 557 cm⁻¹ for all three iMS-OFF nanoprobes after 24-hour incubation with both targets and RTP strands (Target (+) and RTP (+)) were significantly higher than those incubated with targets in the absence of the RTP strands (Target (+) and RTP (−)). The increased intensity (blank-subtracted intensity) was greater when the Placerholder-1 was used (FIG. 8A), compared to the Placeholder-2 (FIG. 8B) and Placeholder-3 (FIG. 8C). In addition, after 3-hour incubation with targets, a better amplification efficiency (i.e., a greater difference between the RTP (+) and RTP (−) samples in the presence of targets) was also found when the Placeholder-1 was used, while the sample with the Placeholder-3 only showed a slight difference with or without RTP strands.

The different amplification efficiencies for the three placeholders shown in Table 1 are affected by the difference in the free energy (ΔG) of the toehold-1 domain. The lower ΔG (−8.8 kcal/mol) observed for the Placeholder-1 and Placeholder-2 demonstrates a better binding strength compared to the ΔG (−7.0 kcal/mol) for the Placeholder-3.

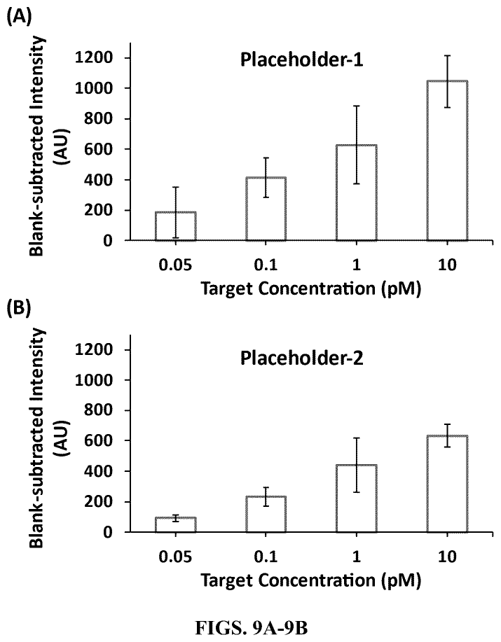

For miR-21 iMS-OFF nanoprobes hybridized with either Placerholder-1 or Placerholder-2 and incubated with target and RTP strand, FIG. 9 shows the blank-subtracted SERS intensity at 557 cm⁻¹ is increased as the target concentration is increased from 50 fM to 10 PM for both Placeholder-1 (FIG. 9A) and Placeholder-2 (FIG. 9B). However, the increased SERS intensity for the Placeholder-1 at each target concentration was found to be greater than that for the Placeholder-2. This may be caused by the one additional base at the 3′ end of the Placeholder-2, which can affect the dissociation of the placeholders from the DNA probes upon target binding.

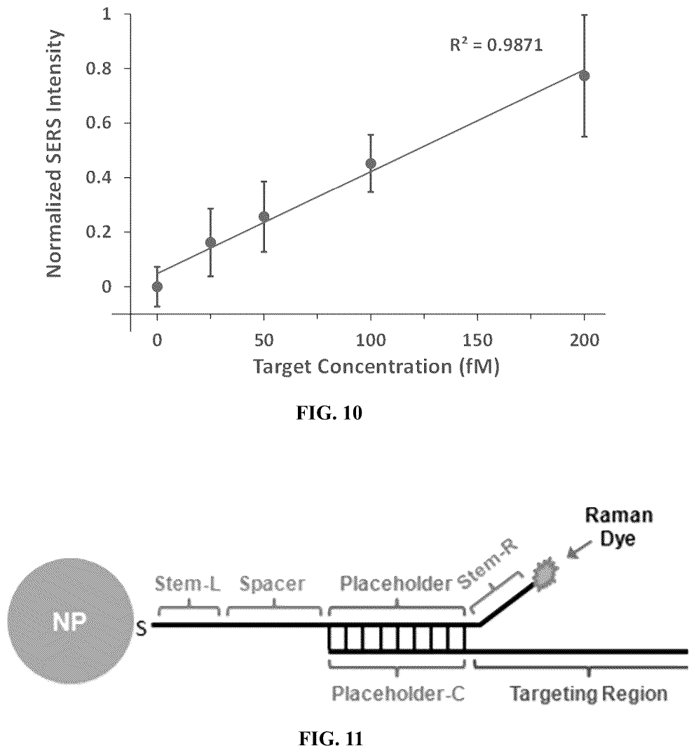

A limit of detection (LOD) can be determined by performing a quantitative analysis using the iMS-OFF nanoprobes with Placeholder-1 for target concentration between 0 and 200 fM. FIG. 10 shows a linear trend line fitted to the data (normalized blank-subtracted SERS intensity at 557 cm⁻¹) for target concentrations at 0, 25, 50, 100, and 200 fM with R2 value of 0.9871. The LOD is determined to be 45 fM based on the 30-rule by using the best-fit linear equation and the standard deviation of the normalized intensity from the blank. This result shows that the CARTP amplification strategy provides a significantly improved detection sensitivity, which is 100-fold more sensitive than the non-amplified iMS assay previously published. These results show that iMS amplification catalyzed by a “linear” RTP strand can be more sensitive than previously published methods for detecting miRNA and other short nucleic acids.

FIG. 11 shows a schematic diagram of an inverse molecular sentinel (iMS) “nanosensor”, “nanoprobe”, “probe”, or “nanoconstruct”. In one embodiment, the iMS nanoprobes, having a Raman label at one end of the stem, are immobilized onto a metallic nanoparticle via a Au-thiol bond formed on the other end of the stem. Because the plasmon field enhancement decreases significantly from the surface, a molecule must be located within a very close range (0-10 nm) of the nanostructure surface in order to experience the enhanced local plasmon field. As shown in FIG. 11, a complementary “capture probe” or “placeholder” strand bound to the nanoconstruct keeps the Raman dye away from the nanoparticle surface; the probe is “open” with low SERS signal, which is the ‘Off’ state. Upon exposure to the target sequence, the capture probe (“placeholder” strand) leaves the nanoprobe based on competitive binding to the target, allowing the stem-loop to “close” and move the Raman label onto the nanoparticle surface. Upon laser excitation, the Raman label molecule experiences a strong plasmonic effect and generates an intense SERS signal, which is the ‘On’ status.

The plasmonic-active nanoparticles of the iMS nanoprobes described herein can include, but are not limited to, silver nanospheres, gold nanospheres, nanospheroids, nanoshells, nanorods, nanowires, nanocubes, nanoprisms, nanopyramids, and nanostars. These nanoparticles can be used to yield an intense SERS signal of the label at different plasmon resonance wavelengths. The thiolated SERS reporter-strand can be designed to have a Raman dye at one end as the reporter, and a thiol group at the other end for attaching to the nanoparticle. In other embodiments, the reporter-strand can be attached to the nanoparticle using any method known to those of skill in the art. In addition, the SERS optical reporter can be selected from, but not limited to, Raman dye, 3,3′-Diethylthiadicarbocyanine iodide (DTDC), 3,3′-diethylthiatricarbocyanine iodide (DTTC), 1,1′,3,3,3′,3′-Hexamethylindotricarbocyanine iodide (HITC), CY3 dye, CY3.5 dye, CY5.5 dye, CY7 dye, CY7.5 dye, a positively-charged hydrophobic near infrared (NIR) dye, IR-780, IR-792, IR-797, IR-813, methylene blue hydrate (MB), 4-mercaptobenzoic acid (4-MBA), 5,5′-dithiobis-2-nitrobenzoic acid (DTNB), 4-aminothiophenol (4ATP), fluorescein, fluorescein isothiocyanate (FITC), thionine dyes, rhodamine-based dye, and crystal violet.

The reporter strand has four segments: stem-L, stem-R, spacer, and placeholder. The stem-L and stem-R segments allow the stem-loop structure to form after the placeholder-strand binds to the target molecule and leaves the nanoconstruct. The spacer segment is designed to provide sufficient distance (over 10 nm) between the Raman dye and nanoparticle surface to reduce the background SERS signal when the probe is open. The placeholder segment (8-15 nucleotides) binds to the placeholder-strand to prevent the formation of the stem-loop structure. The placeholder—strand has two segments: placeholder-C and targeting region. The placeholder-C segment is complementary to the placeholder segment of the reporter-strand and to the target sequences. The targeting region (20-30 nucleotides) is complementary to the target sequences.

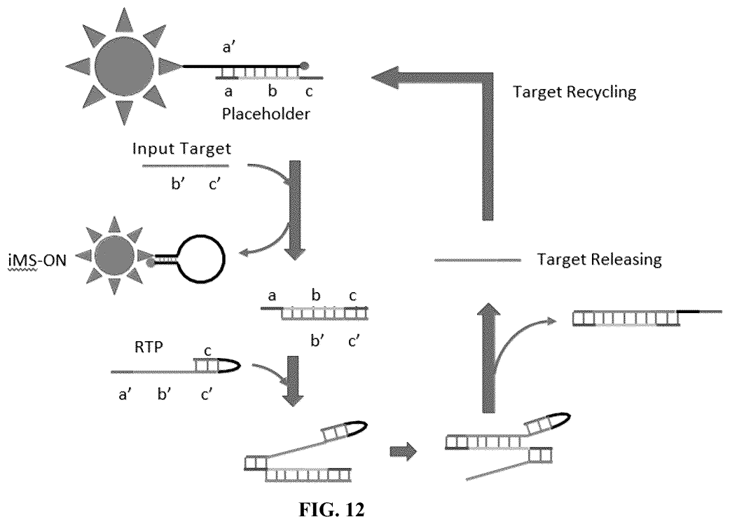

The iMS amplification strategy described herein involves using a Recycling Trigger Probe (RTP). In one embodiment of these aspects, the RTP strand can be designed as a ‘stem-loop” (hairpin) structured strand to prevent the non-specific interaction between the RTP strand and the probe/placeholder duplex in the absence of targets. FIG. 12 shows the iMS amplification strategy using a hairpin RTP strand in the case when the “a” domain of the placeholder is bound to the probe. In FIG. 12, the “a” domain of the placeholder corresponds to “Toehold-2” region and the “c” domain corresponds to the toehold-1 region comprising complementarity to a sequence in a target nucleic acid. In this design, the c’ domain is hidden in the hairpin structure of the RTP stand through the c/c′ stem duplex to prevent the non-specific interaction between the RTP and the iMS probe. In the presence of the target, the target can replace the iMS probe through the strand displacement reaction to produce a target/placeholder duplex. The a′ domain of the hairpin RTP strand can bind to the toehold-2 region (“a” domain) of the target/placeholder duplex to trigger the target recycling process through a second strand displacement reaction. The released target is then reused and recycled to amplify the iMS signal.

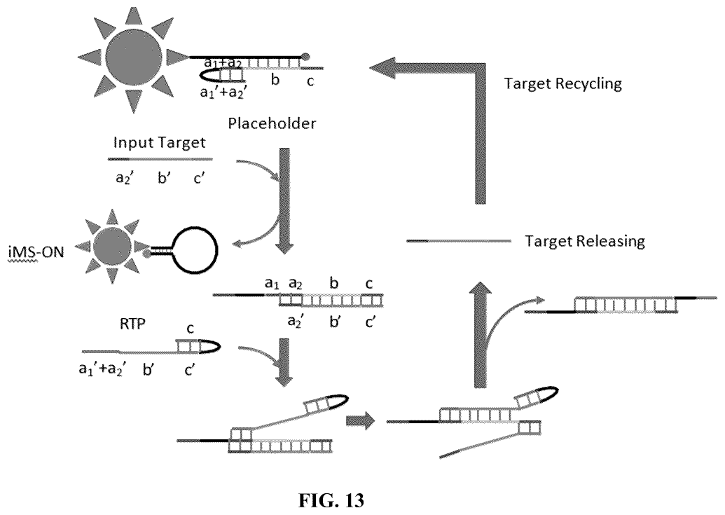

In another embodiment, the placeholder is designed as a hairpin strand with the stem duplex a1+a2/a1′+a2′ (see FIG. 13) to prevent the non-specific interaction with the RTP strand. More specifically, FIG. 13 shows the amplification strategy in the case where the “a” domain of the placeholder is not bound to the probe. In FIG. 13, the “a” domain of the placeholder corresponds to “Toehold-2” region and the “c” domain corresponds to the toehold-1 region comprising complementarity to a sequence in a target nucleic acid. In this strategy, the placeholder is designed as a hairpin strand with the stem duplex a1+a2/a1′+a2′ to prevent the non-specific interaction with the RTP strand in the absence of targets. In the presence of targets, the target can hybridize to the placeholder and open the placeholder loop through the a2′ domain on the target. In this case, the “free” a1 domain on the target/placeholder duplex can serve as the toehold-2 for the RTP strand as the RTP strand has both a1′ and a2′ domains. The RTP strand can then trigger the target recycling process through the second strand displacement reaction.

FIG. 14A is a schematic diagram showing a potential issue with RTP-mediated target recycling and amplification when using a linear RTP strand. FIG. 14B is a schematic diagram showing a toehold locking strategy to prevent the released target from interacting with the placeholder/RTP duplex as illustrated in FIG. 14A by using a “Locker” strand. This toehold locker strand can be used in embodiments described herein. As shown in FIG. 14A, when using a “linear” RTP probe for the iMS amplification strategy, the released target strand has a chance to interact with the placeholder/RTP duplex through the toehold-1 domain instead of being recycled. To address this issue, a toehold locking strategy can be utilized using a “Locker” strand. FIG. 14B shows the toehold locking strategy. In this strategy, the RTP strand has three domains: the a′ domain can bind to the “a” domain (toehold-2) on the target/placeholder duplex to replace the target through b′ domain branch migration. The single-stranded overhang c and d domains on the placeholder/RTP duplex can hybridize to the c′ and d′ domains on the Locker strand. The c′ and d′ domains are designed such that they can only bind to the c and d domains when the placeholder/RTP duplex is formed (i.e., the Locker strand cannot bind to the single-stranded RTP or placeholder strand). In this way, the toehold-1 domain on the placeholder/RTP duplex is locked and no longer available for the released target.

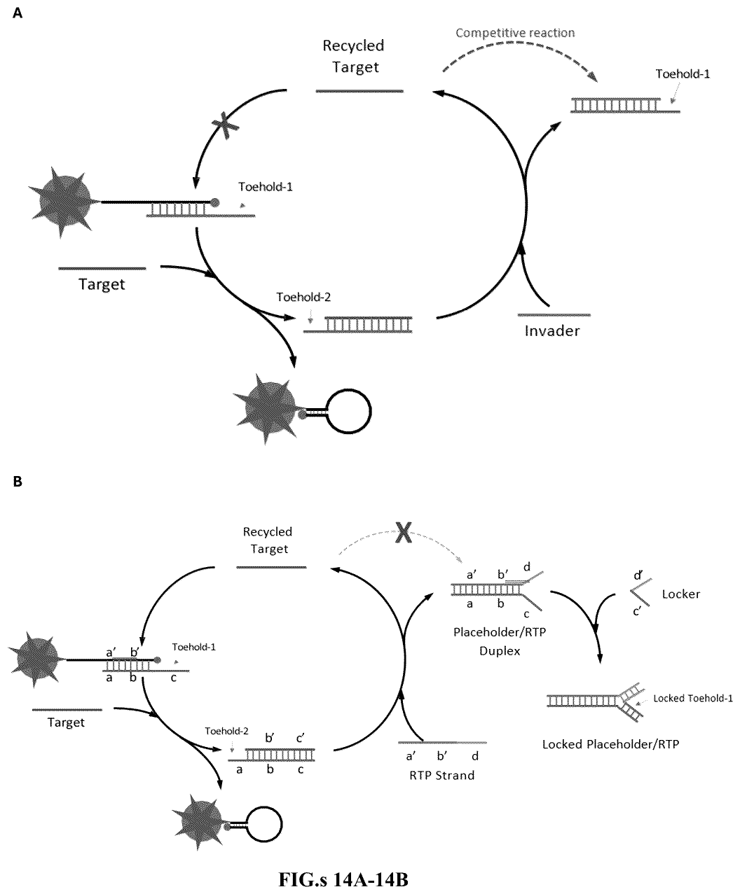

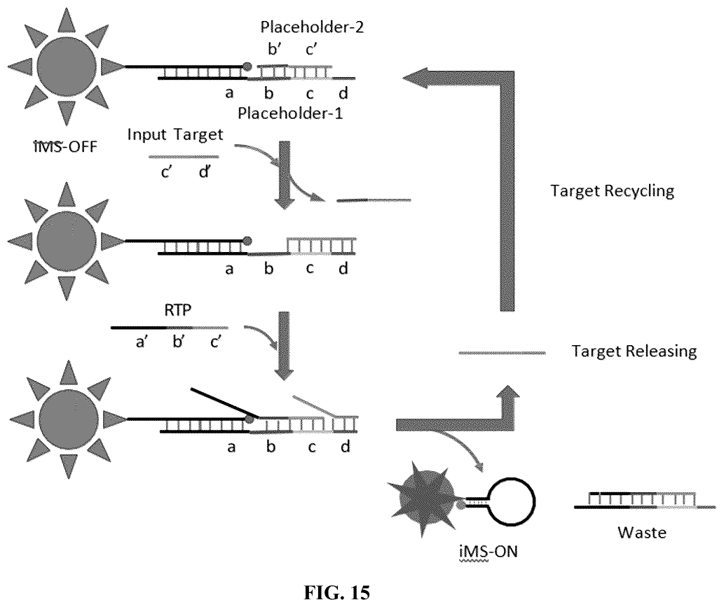

FIG. 15 shows an embodiment of the iMS amplification strategy using a linear RTP strand and two placeholders. In this embodiment, the iMS is turned OFF by hybridizing the probe with the placeholder-1 having 4 domains (a, b, c, and d). The placeholder-1 is also binding with placeholder-2 strand having b′ and c′ domains. In the presence of the target, the target can bind to the d (toehold) and replace the placeholder-2 strand. In this way, the b domain of the placeholder-1 becomes a single-stranded region that can serve as the toehold for the RTP strand allowing the RTP strand to bind to the b domain and trigger the strand displacement reaction. This process can lead to the iMS being turned ON and releasing the target strand for recycling.

A plasmonic-active nanoprobe system for detecting a target nucleic acid is provided. The system comprising: a) at least one plasmonic-active nanoparticle; b) a nucleic acid probe comprising a sequence that forms a stem loop, a first end attached to the at least one plasmonic-active nanoparticle, and a second end labeled with an optical reporter; c) a recycling trigger nucleic acid probe (RTP); d) a first placeholder nucleic acid strand comprising: i) at one end, a first region and a toehold-1 region, each of which is complementary to a sequence in a target nucleic acid, ii) an internal toehold-2 region complementary to a sequence in the RTP, and iii) a fourth region at the opposite end to the first region, complementary to the sequence in the nucleic acid probe that forms a stem-loop or a portion thereof; and e) a second placeholder nucleic acid strand having a region complementary to the toehold-2 region of the first placeholder and a region complementary to the first region of the first placeholder and excluding complementarity to the toehold-1 region. When the first placeholder nucleic acid strand is hybridized with the nucleic acid probe and the second placeholder nucleic acid strand is hybridized with the first placeholder nucleic acid strand, the nucleic acid probe is turned OFF. In the presence of the target nucleic acid, the target binds to the toehold-1 region and thereby replaces the second placeholder nucleic acid strand. The internal toehold-2 region of the first placeholder becomes single stranded and the RTP strand binds to the toehold-2 region thereby releasing the first placeholder nucleic acid strand from the nucleic acid probe. The stem loop of the nucleic acid probe closes, thereby inducing an optical signal from the optical reporter, turning the nucleic acid probe ON, and releasing the target strand for recycling.

A method for detection of a target nucleic acid in a sample is provided. The method comprising contacting a sample with a plasmonic-active nanoprobe system, the system comprising: a) at least one plasmonic-active nanoparticle; b) a nucleic acid probe comprising a sequence that forms a stem loop, a first end attached to the at least one plasmonic-active nanoparticle, and a second end labeled with an optical reporter; c) a recycling trigger nucleic acid probe (RTP); d) a first placeholder nucleic acid strand comprising: i) at one end, a first region and a toehold-1 region, each of which are complementary to a sequence in a target nucleic acid, ii) an internal toehold-2 region complementary to a sequence in the RTP, and iii) a fourth region at the opposite end to the first region, complementary to the sequence in the nucleic acid probe that forms a stem-loop or a portion thereof, and c) a second placeholder nucleic acid strand having a region complementary to the toehold-2 region of the first placeholder and a region complementary to the first region of the first placeholder and excluding complementarity to the toehold-1 region. When the first placeholder nucleic acid strand is hybridized with the nucleic acid probe and the second placeholder nucleic acid strand is hybridized with the first placeholder nucleic acid strand, the nucleic acid probe is turned OFF. The method comprises detecting an optical signal from the optical reporter in the presence of the target nucleic acid. Specifically, in the presence of the target nucleic acid, the target binds to the toehold-1 region and thereby replaces the second placeholder nucleic acid strand. The internal toehold-2 region of the first placeholder becomes single stranded and the RTP strand binds to the toehold-2 region thereby releasing the first placeholder nucleic acid strand from the nucleic acid probe. The stem loop of the nucleic acid probe closes, thereby inducing an optical signal from the optical reporter turning the nucleic acid probe ON and releasing the target strand for recycling.

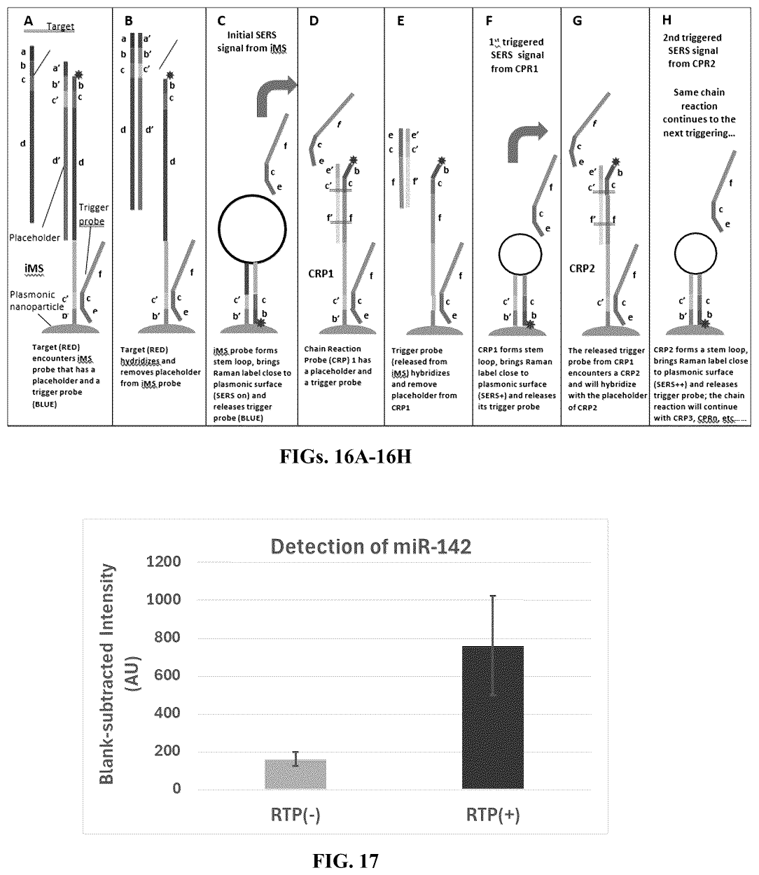

Another aspect of the invention is illustrated in FIG. 16. Specifically, FIG. 16 shows a schematic diagram of a nano-plasmonic molecular probes (NPM) Amplification System Using Chain Reaction Probe (CRP). In a first step, the target encounters the iMS probe that has a placeholder and a trigger probe (see FIG. 16A). Next, the target hybridizes and removes the placeholder from the iMS probe (see FIG. 16B). In the next step, the iMS probe forms a stem loop, brings the Raman label close to the plasmonic surface (SERS on) and releases the trigger probe (see FIG. 16C). Next, the Chain Reaction Probe (CRP) 1 has a placeholder and a trigger probe (see FIG. 16D). In the next step, the Chain Reaction Probe (CRP) 1 has a placeholder and a trigger probe (see FIG. 16E). Subsequently, the CRP1 forms a stem loop, brings the Raman label close to the plasmonic surface (SERS+) and releases its trigger probe (FIG. 16F). Next, the released trigger probe from CRP1 encounters a CRP2 and hybridizes with the placeholder of CRP2 (FIG. 16G). CRP2 forms a stem loop, brings the Raman label close to the plasmonic surface (SERS++) and releases the trigger probe. The chain reaction shown in FIGS. 16A-16G will continue with CRP3 (FIG. 16H). And the chain reaction continues with subsequent CPRs (CPRn, etc.).

A kit is provided for detecting a target nucleic acid, the kit comprising a plasmonic-active nanoprobe system. The plasmonic-active nanoprobe system comprises a) at least one plasmonic-active nanoparticle; b) a nucleic acid probe comprising a sequence that forms a stem loop, a first end attached to the at least one plasmonic-active nanoparticle, and a second end labeled with an optical reporter; c) a recycling trigger nucleic acid probe (RTP); d) a placeholder nucleic acid strand comprising: i) at one end a toehold-1 region comprising complementarity to a sequence in a target nucleic acid, ii) at the other end a “toehold-2” region comprising complementarity to a sequence in the RTP, and iii) a third region between the toehold-1 and toehold-2 regions complementary to the sequence in the nucleic acid probe that forms a stem-loop or a portion thereof; and e) directions for contacting a sample with the plasmonic-active nanoprobe system and detecting an optical signal from the optical reporter in the presence of the target nucleic acid.

In the kit, the RTP can be a linear RTP. In one embodiment, the RTP can comprise a hairpin at the region comprising complementarity to the toehold-1 region of the placeholder nucleic acid strand. In another embodiment, the placeholder nucleic acid strand can comprise a hairpin at the toehold-2 region. In yet another embodiment, the kit can further comprise a locker nucleic acid strand, wherein the locker nucleic acid strand comprises a first domain complementary to an end of the RTP strand opposite to the “toehold-2” region and a second domain complementary to the toehold-1 region of the placeholder nucleic acid strand, wherein the first and second domains of the locker strand only bind to the placeholder/RTP duplex and cannot bind to the single-stranded RTP or placeholder nucleic acid strands.

A kit is provided for detecting a target nucleic acid, the kit comprising a plasmonic-active nanoprobe system that includes two different placeholder strands. Specifically, the plasmonic-active nanoprobe system comprises: a) at least one plasmonic-active nanoparticle; b) a nucleic acid probe comprising a sequence that forms a stem loop, a first end attached to the at least one plasmonic-active nanoparticle, and a second end labeled with an optical reporter; c) a recycling trigger nucleic acid probe (RTP); d) a first placeholder nucleic acid strand comprising: i) at one end, a first region and a toehold-1 region, each of which are complementary to a sequence in a target nucleic acid, ii) an internal toehold-2 region complementary to a sequence in the RTP, and iii) a fourth region at the opposite end to the first region, complementary to the sequence in the nucleic acid probe that forms a stem-loop or a portion thereof; e) a second placeholder nucleic acid strand having a region complementary to the toehold-2 region of the first placeholder and a region complementary to the first region of the first placeholder and excluding complementarity to the toehold-1 region; and f) directions for contacting a sample with the plasmonic-active nanoprobe system and detecting an optical signal from the optical reporter in the presence of the target nucleic acid.

A kit is provided for detecting a protein target. The kit comprising a plasmonic-active nanoprobe system, comprising: a) at least one plasmonic-active nanoparticle; b) a nucleic acid probe comprising a sequence that forms a stem loop, a first end attached to the at least one plasmonic-active nanoparticle, and a second end labeled with an optical reporter; c) a recycling trigger nucleic acid probe (RTP); d) an aptamer placeholder nucleic acid strand comprising: i) at one end a first region comprising a “toehold-1” portion and a portion having a secondary structure that can bind to a target protein, ii) at the other end a “toehold-2” region comprising complementarity to a sequence in the RTP, and iii) a third region between the first and toehold-2 regions complementary to the sequence in the nucleic acid probe that forms a stem-loop or a portion thereof; and e) directions for contacting a sample with the plasmonic-active nanoprobe system and detecting an optical signal from the optical reporter in the presence of the target protein.

### Example 1

Synthesis of SERS-Active iMS Nanoprobes and Testing of Cascade Amplification by Recycling Trigger Probe (CARTP)

Synthesis of silver-coated gold nanostars. The silver-coated gold nanostars (AuNS@Ag) were prepared as described previously. Briefly, 12 nm citrate gold seed solution was first prepared using a modified Turkevich method. Gold nanostars (AuNS) were synthesized by addition, in order, of 50 μL of 6 mM AgNO₃ and 50 μL of 0.1 M ascorbic acid to a solution containing 10 mL of 0.25 mM HAuCl4, 10 μL of 1 N HCl, and 100 μL of the 12 nm gold seed solution under stirring at room temperature. After stirring for 30 seconds, 50 μL of 0.1 M AgNO3 was added to the AuNS solution, followed by 10 μL of 29% NH4OH to initiate the silver coating reaction. The color of the solution changed from blue to purple to dark brown over the course of about 5 minutes.

The obtained solution was used for further functionalization without purification. The stock concentration of nanostars is approximately 0.1 nM, as determined by nanoparticle tracking analysis (NTA).

Synthesis of SERS-active iMS nanoprobes. The iMS nanoprobes were synthesized as described in previous publications with slight modifications according to a pH-assisted method. FIG. 5 shows the schematic diagram illustrating the preparation process for the synthesis of the iMS nanoprobes. To adjust the pH of the prepared AuNS@Ag solution, a citrate buffer containing 0.1 M sodium citrate dihydrate, and 0.3 N HCl was prepared. The stem-loop probes were incubated with 100× molar excess of TCEP (Tris(2-carboxyethyl) phosphine hydrochloride) at room temperature for 1.5 hours to reduce disulfide bonds. The TCEP-treated probe was added to the as-prepared AuNS@Ag (1×) at a final concentration of 0.2 μM. The mixture (0.9 mL) was sonicated for 10 seconds followed by addition of 100 μL of the prepared citrate-HCl buffer. The mixture was then allowed to react at room temperature for 10 minutes followed by addition of 100 μL of 10 μM Thiol-PEG (mPEG-SH, MW 5000). After standing at room temperature for 30 minutes, the solution was mixed with 10 μL of 1% Tween-20, followed by centrifugal washed (9,000 rpm, 10 min) and resuspended in 10 mM Tris-HCl buffer (pH 8.0) containing 0.01% Tween-20. The nanostar surface was then passivated using 0.1 mM 6-mercapto-1-hexanol (MCH) for 10 min at 37° C. followed by four additional centrifugal washing steps (9,000 rpm, 10 min) using Tris-HCl buffer (10 mM, pH 8.0) containing 0.01% Tween-20. After the fourth centrifugation, the pellet was resuspended in 200 μL of 10 mM sodium phosphate buffer containing 0.01% Tween-20.

To prepare iMS-OFF nanoprobes, the iMS-AuNS@Ag solution was incubated with 2 μM placeholder DNA in 1×PBS buffer containing 0.01% Tween-20 at 37° C. for about 20 hours. The excess placeholder strands were removed by four centrifugal washing steps (9,000 rpm, 10 min) and finally resuspended in 1×PBS buffer containing 0.01% Tween-20. The iMS solution was then stored at 4° C. until further use.

iMS Assay Procedure and SERS Measurements. The iMS assay was carried out in triplicate by mixing iMS solution with miR-21 synthetic targets or a mixture of miR-21 synthetic targets and invaders in 1×PBS buffer containing 0.01% Tween-20 and 5 mM MgCl₂. The mixture was allowed to react at room temperature for 2 or 22 hours. Following the reaction, 100 μL of the mixture were used for the SERS measurements in a glass vial using a Renishaw In Via confocal Raman microscope equipped with a 632.8 nm HeNe laser. The light from the laser was focused into the 100 μL sample solution with a 10× microscope objective after passing through a laser line filter. All SERS spectra were background subtracted and smoothed in MATLAB using a Savitsky-Golay filter with five-point window and first-order polynomial. Three SERS measurements were performed per sample and averaged into a single spectrum.

Quantification of Cy5-labeled DNA probes immobilized on silver-coated gold nanostars. The number of the Cy5-labeled oligonucleotides immobilized on AuNS@Ag was determined by a dithiothreitol (DTT)-based ligand displacement assay described previously. This DTT-based assay has been recognized as a “gold standard” for determining the surface coverage of thiolated oligonucleotides on gold nanoparticles. Briefly, 100 μL of Cy5-labeled iMS-OFF nanoprobes (final concentration 0.1 nM) were incubated with DTT (final concentration 0.5 M) in 10 mM Tris-HCl buffer (pH 8.0) containing 0.01% Tween-20 for 20 hours at room temperature with gentle shaking. This ligand exchange process allows to displace Cy5-labeled probes from the nanostar surface completely. The solutions were centrifuged at 9,000 rpm for 10 min to separate the displaced oligonucleotides from nanostars. After centrifugation, aliquots of the supernatant (50 μL) were collected and mixed with 50 μL of 10 mM Tris-HCl buffer containing 0.01% Tween-20. The supernatant mixtures were then transferred into a 96-well microplate to record the fluorescence using the FLUOstar Omega microplate reader (BMG Labtech, Inc.). The collected supernatants were excited at 550 nm and the fluorescent emission was measured at 580 nm. The concentrations of the released Cy5-labeled probes were determined according to a standard curve. Standard curve samples were prepared with known concentrations of the Cy5-labeled oligonucleotides using the same incubation and centrifugation procedures. The average number of oligonucleotides per nanostar was then determined by dividing the measured oligonucleotide concentration by the nanostar concentration in the sample.

Probe Design of CARTP Strategy for Amplification. The cascade iMS amplification is achieved through two toehold-mediated strand displacement reactions. To initiate the amplification process, the target strand first needs to bind to the toehold-1 domain on the probe/placeholder duplex for the first strand displacement reaction. To release the target, the RTP strand needs to bind to the toehold-2 domain on the placeholder/target duplex for the second strand displacement reaction. Table 1 shows the sequences of the oligonucleotides used in this study, including the thiolated Cy5-labeled stem-loop probe, placeholders, RTP strand and target. In previous publications, a 9-base toehold-1 domain was successfully used for the iMS assay without amplification. In this study, three placeholders were tested for the signal amplification strategy. Free energy (ΔG) values of the toehold domains were also calculated using the Two State Melting Hybridization tool on the DINA Melt web server (http://www.unafold.org). To initiate the first displacement reaction, the toehold-1 domain was designed to be 8 bases with ΔG=−8.8 kcal/mol for the Placeholder-1 and Placeholder-2. For the Placeholder-3, the toehold-1 domain was designed to be 7 bases with ΔG=−7.0 kcal/mol (FIG. 6A). To trigger the second strand displacement reaction, a 7-base toehold-2 domain with a moderate binding strength (ΔG=−7.0 kcal/mol) was used for all placeholders (FIG. 6B). Accordingly, the stem-loop probe was modified to have two guanine (C) bases in the internal spacer instead of two adenine (A) bases in the original design from our previous publications. The Placeholder-2 was designed to have one additional base at the 3′ end to increase the stability of the probe/placeholder duplex.

TABLE 1
Exemplary oligonucleotide sequences for stem-
loop probe, placeholder, RTP strand, and
target.

| Name | Sequence (5′→3′) |
|---|---|
| Stem-loop probe* | thiol-AAAAAGTCTGTATACCAAAA |
| | TAGCTTATCAGAC-Cy5 |
| | (SEQ ID NO: 1) |
| Placeholder-1 | CAACATCAGTCTGATAAGCTATTTTG |
| | GT (SEQ ID NO: 2) |
| Placeholder-2** | CAACATCAGTCTGATAAGCTATTTTG |
| | GTA (SEQ ID NO: 3) |
| Placeholder-3** | AACATCAGTCTGATAAGCTATTTTGG |
| | T (SEQ ID NO: 4) |
| RTP strand | ACCAAAATAGCTTATCAGAC |
| | (SEQ ID NO: 5) |
| Target | TAGCTTATCAGACTGATGTTGA |
| | (SEQ ID NO: 6) |

*Sequences in bold represent the modified bases compared to the original design in previous publications.
**Underlined sequences represent the toehold-1 domain for the target strand at the 5′ end and the toehold-2 domain for the RTP strand at the 3′ end.

To demonstrate the iMS amplification strategy, the amplification efficiency was first evaluated using the miR-21 iMS nanoprobes hybridized with three difference placeholders (Placeholder-1, Placeholder-2 and Placeholder-3). The average number of the Cy5-labeled probes immobilized on a AuNS@Ag was estimated to be 680 oligonucleotides per particle, which is 3.4 nM probes in 5 pM iMS-OFF nanostars used in this study. These iMS-OFF nanoprobes were then incubated with 1 nM synthetic DNA targets (denoted as Target (+)) in the presence (denoted as RTP (+)) or absence (denoted as RTP (−)) of 100 nM RTP strands at room temperature for 3 or 24 hours. FIG. 7 presents an example of representative SERS spectrum within the spectral region of the major peak of the iMS nanoprobes with Placeholder-1 in the presence of the RTP strands. The arrow indicates the main Cy5 Raman peak at 557 cm⁻¹ that was used for the peak-height intensity analysis in this study. As shown in FIG. 7A, the SERS intensity at 557 cm⁻¹ was significantly increased after incubation with miR-21 targets, indicating that a “closed” stem-loop probe structure was formed and the SERS signal was turned on upon the binding of the targets. Such a response can be clearly seen in FIG. 7B, which shows the increased SERS intensity after blank subtraction.

FIG. 8 shows that the blank-subtracted SERS peak-height intensities at 557 cm⁻¹ for all three iMS-OFF nanoprobes after 24-hour incubation with both targets and RTP strands (Target (+) and RTP (+)) were significantly higher than those incubated with targets but in the absence of the RTP strands (Target (+) and RTP (−)). The increased intensity (blank-subtracted intensity) was greater when the Placerholder-1 was used (FIG. 8A), compared to the Placeholder-2 (FIG. 8B) and Placeholder-3 (FIG. 8C). In addition, after 3-hour incubation with targets, a better amplification efficiency (i.e., a greater difference between the RTP (+) and RTP (−) samples in the presence of targets) was also found when the Placeholder-1 was used, while the sample with the Placeholder-3 only showed a slight difference with or without RTP strands.

The different amplification efficiencies for these three placeholders are affected by the difference in the free energy (ΔG) of the toehold-1 domain; this free energy has previously been shown to affect the kinetics of the strand displacement. The lower ΔG (−8.8 kcal/mol) observed for the Placeholder-1 and Placeholder-2 demonstrates a better binding strength compared to the ΔG (−7.0 kcal/mol) for the Placeholder-3. To determine the operating ionic strength for the CARTP assay, the iMS sensor response after 3-hour incubation with 1 nM targets and 100 nM RTP strands in a PBS buffer containing 5 mM MgCl₂ was compared to that in the buffer containing 2 mM MgCl₂ (data not shown). A better sensor response was observed in the case of 5 mM MgCl₂. As Placerhoder-1 and Placeholder-2 yielded the best results, they were selected for further quantification studies using the PBS buffer containing 5 mM MgCl₂ as the reaction buffer.

To prevent or minimize the non-specific interaction between the RTP strand and probe/placeholder duplex, the RTP strand used in this study does not contain the complementary sequences to the toehold-1 domain. However, an increased signal was observed for all three placeholders in the blank samples incubated only with the RTP strands (in the absence of targets), indicating the occurrence of a moderate non-specific interaction between the RTP strand and probe/placeholder duplex (data not shown). However, the results presented herein demonstrate the usefuleness of the CARTP strategy for iMS signal amplification.

Next, the detection sensitivity using the CARTP-mediated amplification strategy was investigated. The miR-21 iMS-OFF nanoprobes hybridized with either Placerholder-1 or Placerholder-2 were incubated with 0.05, 0.1, 1 and 10 pM miR-21 synthetic targets in the presence of 100 nM RTP strands for 24 hours at room temperature. FIG. 9 shows the blank-subtracted SERS intensity at 557 cm⁻¹ was increased with increasing the target concentration from 50 fM to 10 pM for both Placeholder-1 (FIG. 9A) and Placeholder-2 (FIG. 9B). However, the increased SERS intensity for the Placeholder-1 at each target concentration was found to be greater than that for the Placeholder-2. This may be caused by the one additional base at the 3′ end of the Placeholder-2, which can affect the dissociation of the placeholders from the DNA probes upon target binding.

To determine the LOD, a quantitative analysis was then performed using the iMS-OFF nanoprobes with the best performing placeholder, i.e., Placeholder-1, for the target concentration between 0 and 200 fM. The experiments were carried out in triplicate with five SERS measurements per sample on different 5 μL aliquots to minimize the variance between experiments. As shown in FIG. 10, a linear trend line was fitted to the data (normalized blank-subtracted SERS intensity at 557 cm⁻¹) for target concentrations at 0, 25, 50, 100, and 200 fM with R2 value of 0.9871. The LOD was then determined to be 45 fM based on the 30-rule by using the best-fit linear equation and the standard deviation of the normalized intensity from the blank. This result shows that the CARTP amplification strategy provides a significantly improved detection sensitivity, which is 100-fold more sensitive than the non-amplified iMS assay used in previously published studies.

These data demonstrate the utility of the CARTP strategy for cascade iMS amplification catalyzed by a “linear” RTP strand. The simplified iMS amplification assay described herein demonstrated a LOD of 45 fM, which provides a 100-fold improved sensitivity compared with the previously published non-amplified iMS assay.

The CARTP strategy was also demonstrated to detect miR-142, which is an important biomarker for various diseases, including cancer, leukemia, systemic sclerosis, Alzheimer's disease, etc. Table 2 shows the sequences used to detect miR-142. FIG. 17 shows that the blank-subtracted SERS peak-height intensities at 557 cm⁻¹ after 1-hour incubation with both target (100 pM) and the RTP strand (RTP (+)) were significantly higher than those incubated with 100 PM target in the absence of the RTP strand (RTP (−)).

TABLE 2
Exemplary oligonucleotide sequences of stem-
loop probe, placeholder, RTP strand, and
target to detect miR-142-3p miRNA

| Name | Sequence (5′→3′) |
|---|---|
| Stem-loop probe | thiol-AAAAATAGGAATAAAAA |
| | CCATGTAGTGTTTCCTA-Cy5 |
| | (SEQ ID NO: 7) |
| Placeholder | TCCATAAAGTAGGAAACACTACA |
| | TGGTTTT (SEQ ID NO: 8) |
| RTP strand | AAAACCATGTAGTGTTTCCT |
| | A (SEQ ID NO: 9) |
| Target | TGTAGTGTTTCCTACTTTATG |
| | GA (SEQ ID NO: 10) |

One skilled in the art will readily appreciate that the present disclosure is well adapted to carry out the objects and obtain the ends and advantages mentioned, as well as those inherent therein. The disclosure described herein as representative of preferred embodiments, is exemplary, and is not intended as a limitation on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. It will be understood that, unless otherwise stated, reference to any document herein does not constitute an admission that any of these documents form part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

1. A plasmonic-active nanoprobe system for detecting a target nucleic acid, comprising:
at least one plasmonic-active nanoparticle;
a nucleic acid probe comprising a sequence that forms a stem loop, a first end attached to the at least one plasmonic-active nanoparticle, and a second end labeled with an optical reporter;
a recycling trigger nucleic acid probe (RTP); and
a placeholder nucleic acid strand comprising:
at one end a “toehold-1” region comprising complementarity to a sequence in a target nucleic acid,
at the other end a “toehold-2” region comprising complementarity to a sequence in the RTP, and
a third region between the toehold-1 and toehold-2 regions complementary to the sequence in the nucleic acid probe that forms a stem-loop or a portion thereof,
wherein, when the placeholder nucleic acid strand is hybridized with the nucleic acid probe, the optical reporter is at a greatest distance from the plasmonic-active nanoparticle and the nucleic acid probe is turned OFF,
wherein, in the presence of the target nucleic acid, the placeholder nucleic acid strand binds to the sequence in the target nucleic acid, thereby forming a target/placeholder duplex and releasing the nucleic acid probe, wherein the stem loop of the nucleic acid probe closes, thereby inducing an optical signal from the optical reporter and the nucleic acid probe is turned ON, and
wherein the RTP strand binds to the toehold-2 domain of the target/placeholder duplex, thereby forming a placeholder/RTP duplex and releasing the target nucleic acid from the target/placeholder duplex for recycling.

2. The system of claim 1, wherein the RTP is a linear RTP.

3. The system of claim 1, wherein the RTP comprises a hairpin at the region comprising complementarity to the toehold-1 region of the placeholder nucleic acid strand.

4. The system of claim 1, wherein the placeholder nucleic acid strand comprises a hairpin at the toehold-2 region.

5. The system of claim 1, further comprising a locker nucleic acid strand, wherein the locker nucleic acid strand comprises a first domain complementary to an end of the RTP strand opposite to the “toehold-2” region and a second domain complementary to the toehold-1 region of the placeholder nucleic acid strand, wherein the first and second domains of the locker strand only bind to the placeholder/RTP duplex and cannot bind to the single-stranded RTP or placeholder nucleic acid strands.

6. The system of claim 1, wherein the at least one plasmonic-active nanoparticle comprises a silver nanosphere, a gold nanosphere, a nanospheroid, a nanoshell, a nanorod, a nanowire, a nanocube, a nanoprism, a nanopyramid, or a nanostar, and combinations thereof.

7. The system of claim 1, wherein the optical reporter is selected from the group consisting of: Raman dye, 3,3′-Diethylthiadicarbocyanine iodide (DTDC), 3,3′-diethylthiatricarbocyanine iodide (DTTC), 1,1′,3,3,3′,3′-Hexamethylindotricarbocyanine iodide (HITC), CY3 dye, CY3.5 dye, CY5.5 dye, CY7 dye, CY7.5 dye, a positively-charged hydrophobic near infrared (NIR) dye, IR-780, IR-792, IR-797, IR-813, methylene blue hydrate (MB), 4-mercaptobenzoic acid (4-MBA), 5,5′-dithiobis-2-nitrobenzoic acid (DTNB), 4-aminothiophenol (4ATP), fluorescein, fluorescein isothiocyanate (FITC), thionine dyes, rhodamine-based dye, and crystal violet.

8. The system of claim 1, wherein the optical reporter is a Raman dye.

9. The system of claim 1, wherein the optical signal is a Raman signal or surface-enhanced Raman scattering (SERS) signal.

10. The system of claim 1, wherein the nucleic acid target comprises a microRNA, a small noncoding RNA, an mRNA, or a DNA sequence.

11. A method for detection of a target nucleic acid in a sample, comprising:
contacting a sample with a plasmonic-active nanoprobe system, the system comprising:
at least one plasmonic-active nanoparticle,
a nucleic acid probe comprising a sequence that forms a stem loop, a first end attached to the at least one plasmonic-active nanoparticle, and a second end labeled with an optical reporter,
a recycling trigger nucleic acid probe (RTP), and
a placeholder nucleic acid strand comprising:
at one end a “toehold-1” region comprising complementarity to a sequence in a target nucleic acid,
at the other end a “toehold-2” region comprising complementarity to a sequence in the RTP, and
a third region between the toehold-1 and toehold-2 regions complementary to the sequence in the nucleic acid probe that forms a stem-loop or a portion thereof,
wherein, when the placeholder nucleic acid strand is hybridized with the nucleic acid probe, the optical reporter is at a greatest distance from the plasmonic-active nanoparticle and the nucleic acid probe is turned OFF; and
detecting an optical signal from the optical reporter in the presence of the target nucleic acid,
wherein, in the presence of the target nucleic acid, the placeholder nucleic acid strand binds to the sequence in the target nucleic acid, thereby forming a target/placeholder duplex and releasing the nucleic acid probe, wherein the stem loop of the nucleic acid probe closes, thereby inducing an optical signal from the optical reporter and the nucleic acid probe is turned ON, and
wherein the RTP strand binds to the toehold-2 domain of the target/placeholder duplex, thereby forming a placeholder/RTP duplex and releasing the target nucleic acid from the target/placeholder duplex for recycling.

12. The method of claim 11, wherein the nucleic acid target comprises a microRNA, a small noncoding RNA, an mRNA, or a DNA sequence.

13. The method of claim 11, wherein the RTP is a linear RTP.

14. The method of claim 11, wherein the RTP comprises a hairpin at the region comprising complementarity to the toehold-1 region of the placeholder nucleic acid strand.

15. The method of claim 11, wherein the placeholder nucleic acid strand comprises a hairpin at the toehold-2 region.

16. The method of claim 11, wherein the plasmonic-active nanoprobe system further comprises a locker nucleic acid strand, wherein the locker nucleic acid strand comprises a first domain complementary to an end of the RTP strand opposite to the “toehold-2” region and a second domain complementary to the toehold-1 region of the placeholder nucleic acid strand, wherein the first and second domains of the locker strand only bind to the placeholder/RTP duplex and cannot bind to the single stranded RTP or placeholder nucleic acid strands.

17. The method of claim 11, wherein the at least one plasmonic-active nanoparticle comprises a silver nanosphere, a gold nanosphere, a nanospheroid, a nanoshell, a nanorod, a nanowire, a nanocube, a nanoprism, a nanopyramid, or a nanostar, and combinations thereof.

18. The method of claim 11, wherein the optical signal is a Raman signal or surface-enhanced-Raman scattering (SERS) signal.

19. The method of claim 11, wherein the optical reporter is selected from the group consisting of: Raman dye, 3,3′-Diethylthiadicarbocyanine iodide (DTDC), 3,3′-diethylthiatricarbocyanine iodide (DTTC), 1,1′,3,3,3′,3′-Hexamethylindotricarbocyanine iodide (HITC), CY3 dye, CY3.5 dye, CY5.5 dye, CY7 dye, CY7.5 dye, a positively-charged hydrophobic near infrared (NIR) dye, IR-780, IR-792, IR-797, IR-813, methylene blue hydrate (MB), 4-mercaptobenzoic acid (4-MBA), 5,5′-dithiobis-2-nitrobenzoic acid (DTNB), 4-aminothiophenol (4ATP), fluorescein, fluorescein isothiocyanate (FITC), thionine dyes, rhodamine-based dye, and crystal violet.

20. The method of claim 11, wherein the optical reporter is a Raman dye.

21. The method of claim 11, wherein the nucleic acid probe is bound to a substrate.

22. A method for detection of a target protein in a sample, comprising:
contacting a sample with a plasmonic-active nanoprobe system, the system comprising:
at least one plasmonic-active nanoparticle,
a nucleic acid probe comprising a sequence that forms a stem loop, a first end attached to the at least one plasmonic-active nanoparticle, and a second end labeled with an optical reporter,
a recycling trigger nucleic acid probe (RTP), and
an aptamer placeholder nucleic acid strand comprising:
at one end a first region comprising a “toehold-1” portion and a portion having a secondary structure that can bind to a target protein,
at the other end a “toehold-2” region comprising complementarity to a sequence in the RTP, and
a third region between the first and toehold-2 regions complementary to the sequence in the nucleic acid probe that forms a stem-loop or a portion thereof,
wherein, when the aptamer placeholder strand is hybridized with the nucleic acid probe, the optical reporter is at a greatest distance from the plasmonic-active nanoparticle and the nucleic acid probe is turned OFF; and
detecting an optical signal from the optical reporter in the presence of the target protein,
wherein, in the presence of the target protein, the aptamer placeholder strand binds to the target protein, thereby forming a protein/aptamer complex and releasing the nucleic acid probe, wherein the stem loop of the nucleic acid probe closes, thereby inducing an optical signal from the optical reporter and the nucleic acid probe is turned ON, and wherein the RTP strand binds to the toehold-2 domain of the protein/aptamer complex, thereby forming an aptamer placeholder/RTP duplex and releasing the target protein from the protein/aptamer complex for recycling.

23. The method of claim 22, wherein the at least one plasmonic-active nanoparticle comprises a silver nanosphere, a gold nanosphere, a nanospheroid, a nanoshell, a nanorod, a nanowire, a nanocube, a nanoprism, a nanopyramid, or a nanostar, and combinations thereof.

24. The method of claim 22, wherein the optical signal is a Raman signal or surface-enhanced-Raman scattering (SERS) signal.

25. The method of claim 22, wherein the optical reporter is selected from the group consisting of: Raman dye, 3,3′-Diethylthiadicarbocyanine iodide (DTDC), 3,3′-diethylthiatricarbocyanine iodide (DTTC), 1,1′,3,3,3′,3′-Hexamethylindotricarbocyanine iodide (HITC), CY3 dye, CY3.5 dye, CY5.5 dye, CY7 dye, CY7.5 dye, a positively-charged hydrophobic near infrared (NIR) dye, IR-780, IR-792, IR-797, IR-813, methylene blue hydrate (MB), 4-mercaptobenzoic acid (4-MBA), 5,5′-dithiobis-2-nitrobenzoic acid (DTNB), 4-aminothiophenol (4ATP), fluorescein, fluorescein isothiocyanate (FITC), thionine dyes, rhodamine-based dye, and crystal violet.

26. The method of claim 22, wherein the optical reporter is a Raman dye.
